(19) United States
(12) Patent Application Publication
(10) Pub. No.: US 2006/0287529 A1
(43) Pub. Date: Dec. 21, 2006

(54) Solid phase synthesis of antitumoral compounds

(76) Inventors: Mercedes Alvarez, Barcelona (ES); Fernando Albericio, Barcelona (ES); Pablo Cironi, Barcelona (ES); Carmen Cue Vas, Colmenar Viejo Madrid (ES); Andreas Francesch, Madrid (ES); Marta Marfil, Madrid (ES)

Correspondence Address: FISH & RICHARDSON PC, P.O. BOX 1022, MINNEAPOLIS, MN, 55440-1022, US

(21) Appl. No.: 10/544,968

(22) PCT Filed: Feb. 20, 2004
(86) PCT No.: PCT/GB04/00683

§ 371 Date: May 30, 2006

(30) Foreign Application Priority Data
Feb. 20, 2003 (GB) 0303940.1

(51) Int. Cl. C07D 491/04

(52) U.S. Cl. 546/47

(57) ABSTRACT

Lamellarins are prepared using a solid phase synthesis. In a first aspect, the process includes a step as follows: Formula (I) where R₁ to R₁₅ are as defined, and X is halogen, provided that one of R₂, R₃ or R₄ is immobilised to a resin. In a second aspect, the process includes a step as follows: Formula (II) where R₁′ to R₅′, R₁₁′ and R₁₃′ are as defined, X₁ and X₂ are halogen, M is a metal function and PG is an amino protecting group, provided that one of that R₂′, R₃′ or R₄′ is immobilised to a resin.

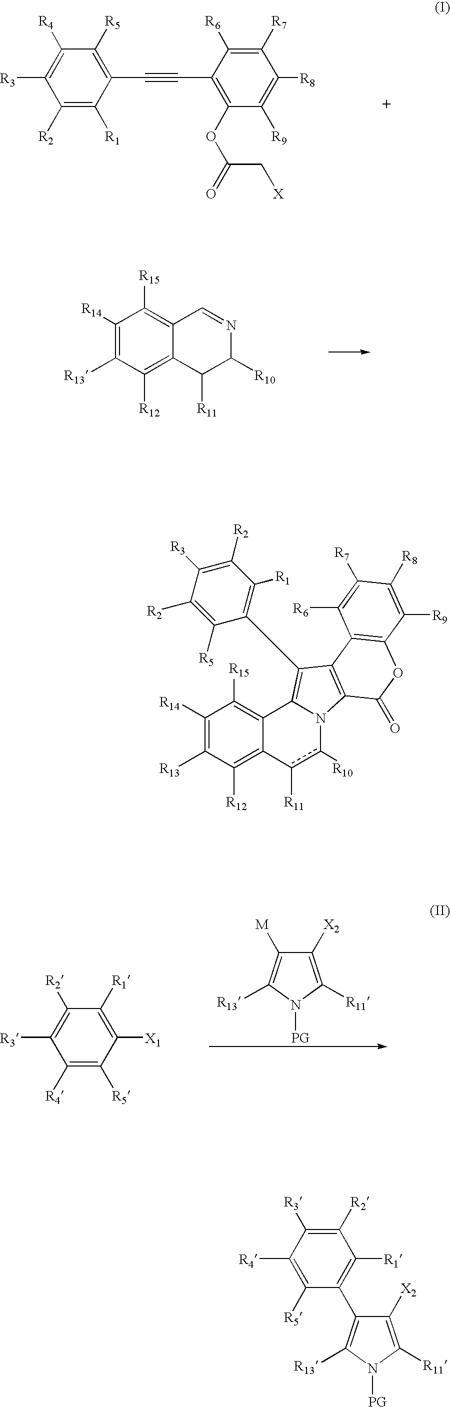

### FIELD OF THE INVENTION

The present invention relates to the field of synthetic chemistry, in particular to the synthesis of useful antitumoral compounds. More particularly, it relates to the solid phase synthesis of lamellarin and related compounds.

### BACKGROUND OF THE INVENTION

The lamellarins are polyaromatics alkaloids originally isolated from marine sources and comprising a fused polyaromatic framework. The family of lamellarins are constituted by two basic structures:

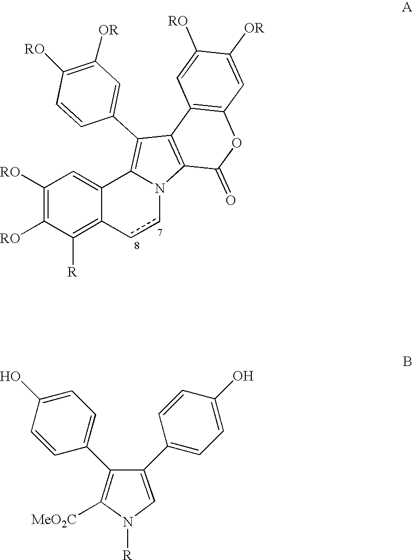

Both structures have a pyrrolic ring substituted with aryl units. Structure A has rings fused to a pyrrole and a ring depending from the the pyrrole. Structure B has rings depending from a pyrrole. The hexacyclic structure A is a 13-phenyl-6H-[1]benzopyran[4′,3′:4,5]pyrrolo[2,1-α]isoquinolin-6-one. Depending on the substituents and the presence of a possible double bond between C₇-C₈, the members of this family are designed with different letters.

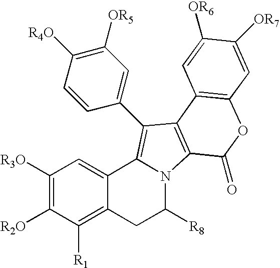

| | R₁ | R₂ | R₃ | R₄ | R₅ | R₆ | R₇ | R₈ |
|---|---|---|---|---|---|---|---|---|
| A | OCH₃ | CH₃ | CH₃ | H | CH₃ | CH₃ | H | OH |
| C | OCH₃ | CH₃ | CH₃ | H | CH₃ | CH₃ | H | H |
| E | OH | CH₃ | CH₃ | CH₃ | H | CH₃ | H | H |
| F | OH | CH₃ | CH₃ | CH₃ | CH₃ | CH₃ | H | H |
| G | H | H | CH₃ | CH₃ | H | H | CH₃ | H |
| I | OCH₃ | CH₃ | CH₃ | CH₃ | CH₃ | CH₃ | H | H |
| J | H | H | CH₃ | CH₃ | CH₃ | CH₃ | H | H |
| K | OH | CH₃ | CH₃ | H | CH₃ | CH₃ | H | H |
| L | H | H | CH₃ | CH₃ | H | CH₃ | H | H |
| S | H | H | CH₃ | H | H | H | H | H |
| T | OCH₃ | CH₃ | CH₃ | CH₃ | H | CH₃ | H | H |
| U | H | CH₃ | CH₃ | CH₃ | H | CH₃ | H | H |
| V | OCH₃ | CH₃ | CH₃ | CH₃ | H | CH₃ | H | OH |
| Y | H | CH₃ | H | CH₃ | H | CH₃ | SO₃Na | H |
| Z | H | H | CH₃ | H | H | H | CH₃ | H |
| β | H | H | H | CH₃ | H | H | H | H |

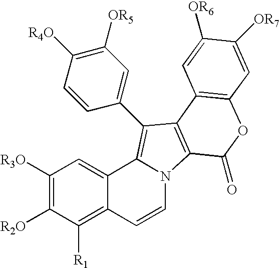

| | R₁ | R₂ | R₃ | R₄ | R₅ | R₆ | R₇ |
|---|---|---|---|---|---|---|---|
| B | OCH₃ | CH₃ | CH₃ | H | CH₃ | CH₃ | H |
| D | H | H | CH₃ | H | CH₃ | CH₃ | H |
| H | H | H | H | H | H | H | H |
| M | OH | CH₃ | CH₃ | H | CH₃ | CH₃ | H |
| N | H | H | CH₃ | CH₃ | H | CH₃ | H |
| W | OCH₃ | CH₃ | CH₃ | CH₃ | H | CH₃ | H |
| X | OH | CH₃ | CH₃ | CH₃ | H | CH₃ | H |
| α | H | CH₃ | CH₃ | CH₃ | H | CH₃ | SO₃Na |

The isolation and characterization of four polyaromatic metabolites gave the lamellarins A-D, obtained from a marine prosobranch mollusc Lamellaria sp. The structure of lamellarin A was determined by an X-Ray crystallographic study and the structures of lamellarins B-D were assigned by interpretation of spectral data.

Four new lamellarins: E-H were isolated and characterized from the marine ascidian Didemnum chartaceum obtained from the Indian Ocean. The structure of lamellarin E was determined by an X-Ray crystallographic study.

Six new lamellarins: I, J, K, L, M and the triacetate of the lamellarin N, and four known compounds of this type: A, B, C, and the triacetate of lamellarin D, were isolated from a marine ascidian Didemnum sp.

Four new lamellarins, O, P, Q, R, with the substructure type B, were isolated and characterized from the marine sponge Dendrilla cactos. Later, the structure of these lamellarins from the ascidian Didemnum sp. was described:

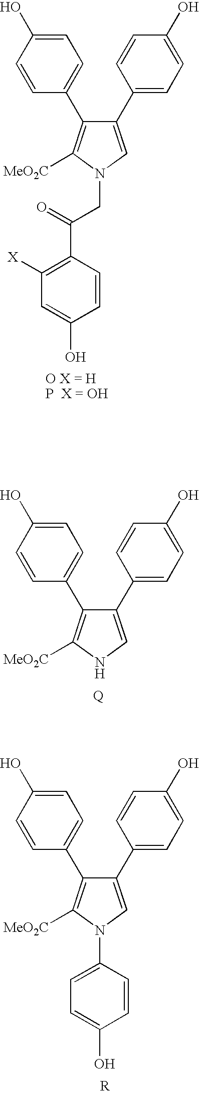

Five new lamellarins: T, U, V, W, and X, and the first example of sulfated lamellarin, Y, were isolated from the marine ascidian Didemnum sp obtained from the Arabian sea.

Lamellarin Z, and various examples of sulphated lamellarins were isolated from the marine ascidian Didemnum chartaceum.

Lamellarin α was isolated from the marine ascidian Didemnum sp.

Lamellarin β obtained from a marine ascidian Didemnum sp.

Lamellarins C and D have been shown to cause inhibition of cell division in a fertilised sea urchin assay, whereas lamellarins I, K, and L exhibit comparable cytoxicity against P388 and A549 cell lines in culture. Recently, lamellarin N has been shown to exhibit activity in lung cancer cell lines by acting as a Type IV microtubule poison.

Furthermore, WO 97/01336 describes that these compounds have also cytotoxic activity on multidrug resistant cells as well as efficacy as non-toxic modulators of the multidrug resistant phenotype and, therefore, afford an attractive potential source of chemotherapeutic agents.

The limited availability of natural material has resulted in the search for alternative synthetic methods being sought for the natural compounds and related analogs. A biomimetic sequence has been described for the synthesis of lamellarin G trimethyl ether by a sequential double cyclization of a 1,3,4-triaryl-2,5-dicarboxysubstituted pyrrole ring. Following this strategy, the synthesis of lamellarin L was achieved.

Another approach has included N-ylide-mediated pyrrole ring formation to install the pyrrole and lactone portions of the lamellarin. This strategy was followed to synthesise lamellarins D and H.

The synthesis of lamellarin K via 1,3-dipolar cycloaddition between an alkyne and an N-ylide of isoquinoline has been reported.

Lamellarin G trimethyl ether was also synthesised. The synthesis involved the formation of the core pyrrolo[2,1-α]isoquinoline, followed by the formation of the lactone ring. The synthesis of lamellarins K and L by the Michael addition/Ring-Closure reaction of benzyldihydroisoquinoline derivatives with ethoxycarbonyl-β-nitrostyrenes has been described.

Lamellarins K and I were obtained by a new approach based on the 1,3-dipolar cycloaddition of a nitrone to an alkyne. The key cycloaddition yield an isoxazoline which rearranged to afford the central pyrrole ring.

In view of the interest for these compounds, their biological properties and their potential as antitumoral compounds, it appears important to provide a process for the preparation of a variety of natural and synthetic lamellarins in an efficient way.

### SUMMARY OF THE INVENTION

The present invention is directed to a process for the solid phase synthesis of lamellarin compounds. This process is also useful for the preparation of libraries of compounds using combinatorial methods.

In one aspect, the present invention provides a process for preparing a lamellarin having rings fused to a pyrrole ring, using solid phase synthesis to form the pyrrole ring. In another aspect, the invention provides a process for preparing a lamellarin having a ring dependent from a pyrrole ring, using solid phase synthesis to link the dependent ring to the pyrrole ring.

According to the first aspect, the process is as follows:

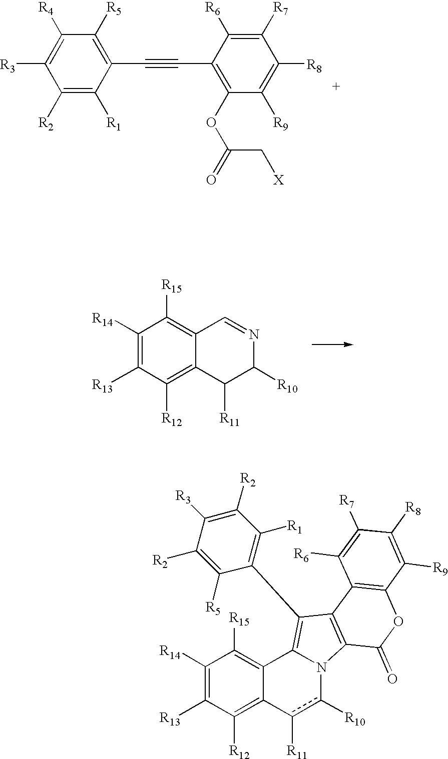

where R₁ to R₁₅ are as defined, and X is halogen, provided that one of R₂, R₃ or R₄ is immobilised to a resin.

According to a second aspect, the process is as follows:

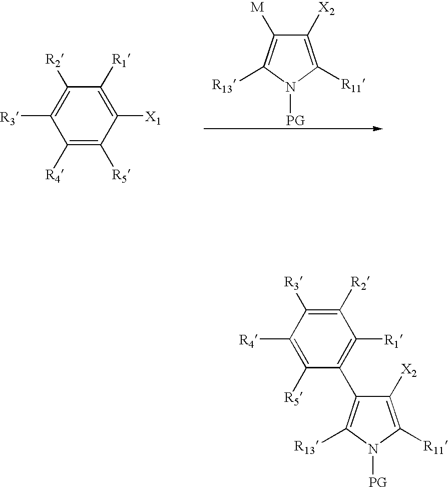

where R₁′ to R₅′, R₁₁′ and R₁₃′ are as defined, X, X₁ and X₂ are halogen, M is a metal function and PG is an amino protecting group, provided that one of R₂′, R₃′ or R₄′ is immobilised to a resin.

In particular, the present invention is directed to a process for the solid phase synthesis of polyheterocyclic structures of formula 1 or 2.

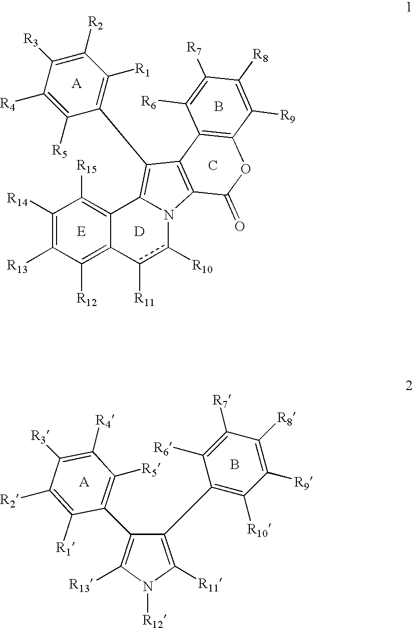

wherein rings A, B and E are preferably aromatic carbocyclic or heterocyclic rings, or with the possible substitution of one or more of these rings for non aromatic heterocyclic rings or for substituted carbocyclic structures;

wherein R₁—R₁₅, R₁′—R₁₁′ and R₁₃′ groups are each independently selected from the group consisting of H, halogen, OH, OR′, SH, SR′, SOR′, SO₂R′, NHR′, N(R′)₂, NHCOR′, N(COR′)₂, NHSO₂R′, OC(═O)H, OC(═O)R′, COOH, COOR′, C₁-C₁₂ alkyl, C₁-C₁₂ haloalkyl, C₂-C₁₂ alkenyl, C₂-C₁₂ alkynyl, substituted or unsubstituted aryl, substituted or unsubstituted aralkyl and substituted or unsubstituted heteroaromatic;

wherein R₁₂′ is selected from the group consisting of H, C₁-C₁₂ alkyl, C₁-C₁₂ haloalkyl, C₂-C₁₂ alkenyl, C₂-C₁₂ alkynyl, substituted or unsubstituted aryl, substituted or unsubstituted arylalkyl and substituted or unsubstituted heteroaromatic;

wherein each of the R′ groups is independently selected from the group consisting of H, OH, NO₂, NH₂, SH, CN, halogen, ═O, C(═O)H, C(═O)CH₃, CO₂H, substituted or unsubstituted C₁-C₁₈ alkyl, substituted or unsubstituted C₂-C₁₈ alkenyl, substituted or unsubstituted C₂-C₁₈ alkynyl, substituted or unsubstituted aryl; and

wherein the dotted line means an optional double bond.

In particular, the present invention is directed to a process for the solid phase synthesis of lamellarin compounds that comprises the step of alkylation of the resin IV with a 3,4-dihydroisoquinoline V followed by a dipolar (2+3) cycloaddition to afford a resin pentacyclic system:

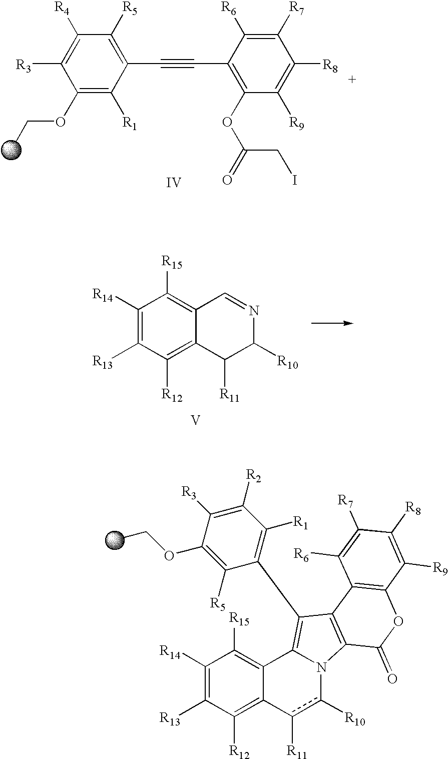

wherein R₁ to R₁₅ are as defined above and iodine shown I is the preferred halogen X.

The invention is also directed to a process for the solid phase synthesis of lamellarin compounds that comprises the step of coupling a resin attached phenol I with 11 to afford the resin VII:

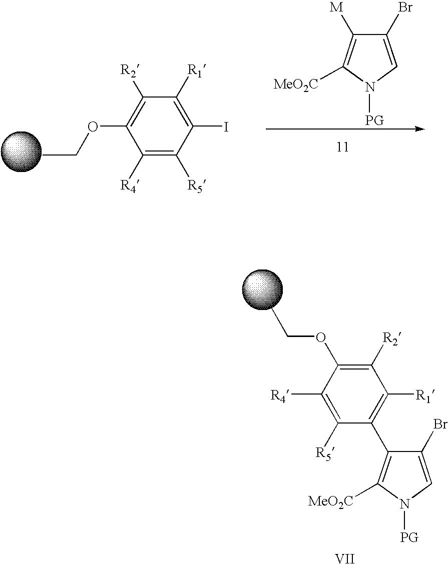

wherein R₁′ to R₅′, M and PG are as defined above, iodine shown I is the preferred halogen X₁ and bromide is the preferred halogen X₂, —CO₂Me is the preferred group for R_{13′}, and hydrogen is the preferred group for R_{11′}. The metal function is of the kind known to the art for achieving the desired reaction, and is preferably chosen from ZnCl, SnBu₃, SnMe₃ or B(MeO)₃, most preferably ZnCl.

### DETAILED DESCRIPTION OF THE INVENTION

Two different procedures for the solid phase synthesis of compounds of structure 1 and 2 are described.

According to one aspect, a step of this invention involves the reaction:

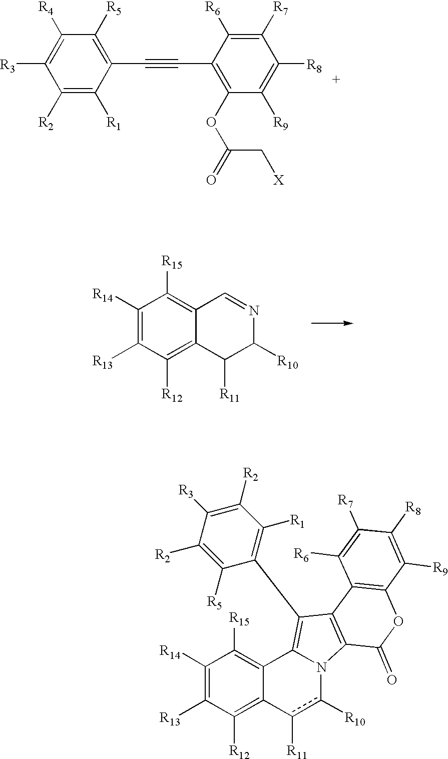

The product can be cleaved from the solid phase to give directly a lamellarin product, or converted by one or more further steps to a lamellarin product.

The step can be part of a scheme as follows:

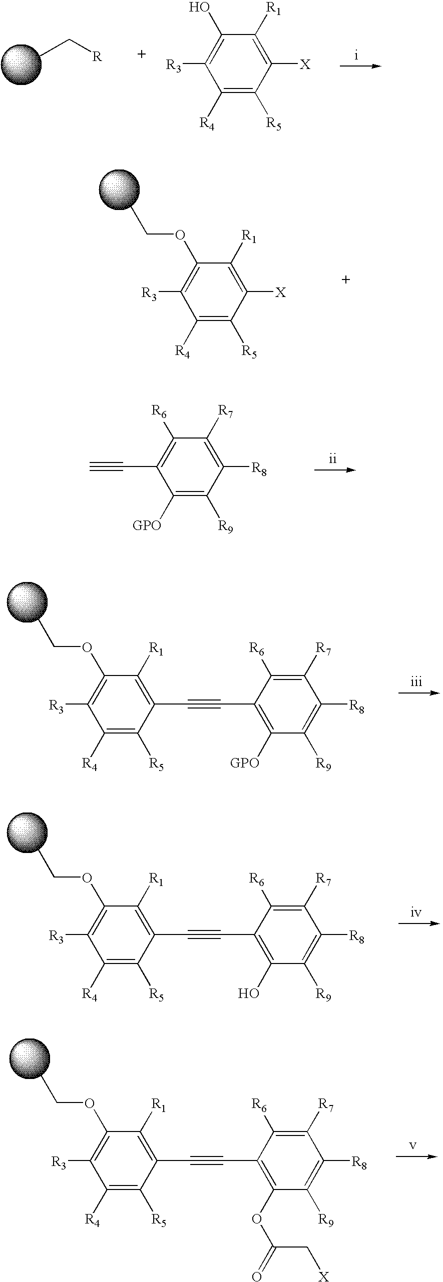

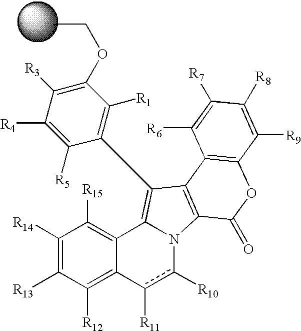

where the solid phase substituent can alternatively be at the positions of R₃ or R₄ instead.

For step (i), a resin with a reactive group R such as —OH or —X is reacted to give a solid phase phenol. In step (ii), the bisarylacetylene compound is obtained by a coupling reaction of the anchored halophenol with an arylacetylene of the appropriate formula. In step (iii), the deprotected aryl acetylene is prepared. In step (iv), the haloacetate is prepared by reaction haloacetic acid. For step (v), alkylation with a 3,4-dihydroisoquinoline followed by a dipolar (2+3) cycloaddition affords the pentacyclic system. Cleavage reaction of the conjugated resin liberates the polycyclic compound of general structure 1. A double bond for the dotted line can be formed as part of step (v) or introduced as a subsequent step.

According to a second aspect, a step of this invention involves the reaction:

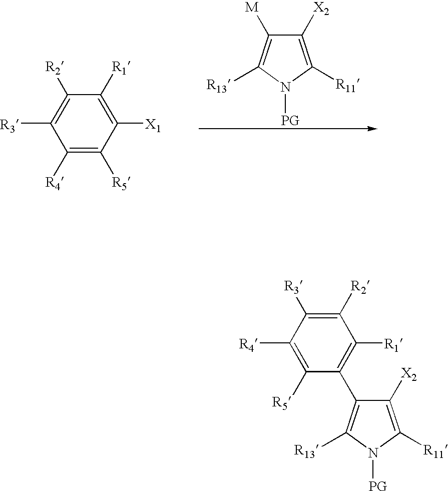

This step can be part of a scheme as follows:

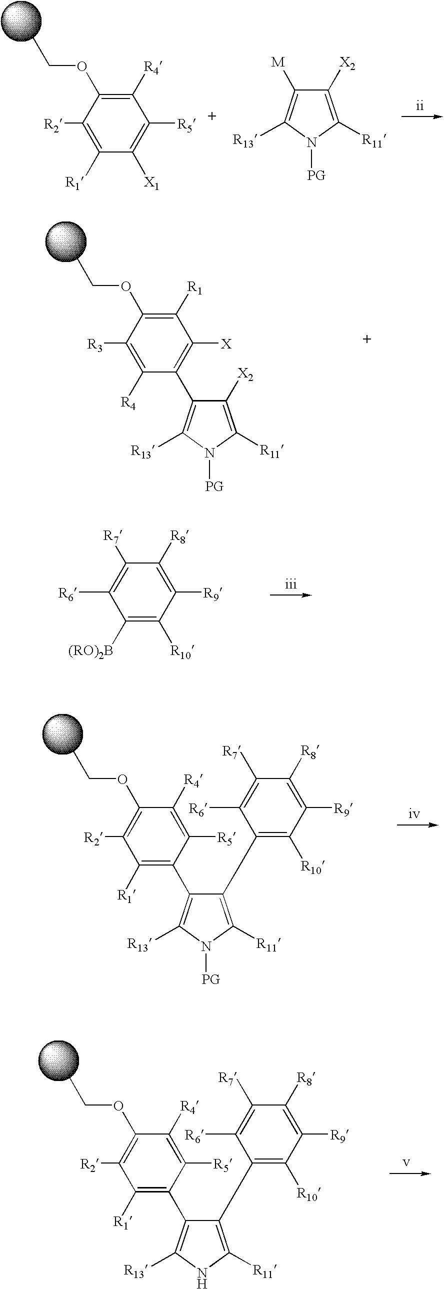

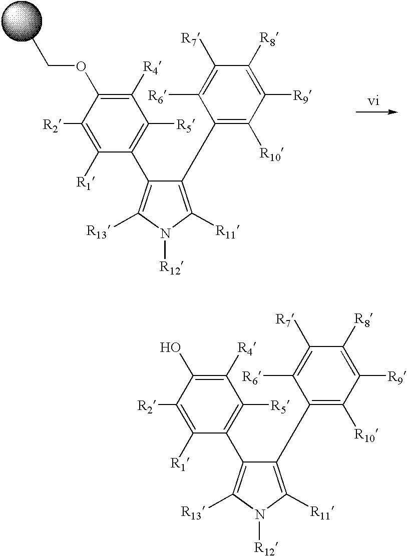

where the solid phase substituent can alternatively be at the positions of R₂′ or R₄′ instead. A solid phase phenol compound is prepared according to step (i) of the first reaction scheme. Step (ii) of the present scheme is the step of the present invention, where the solid phase phenol is reacted with an organometallic compound. In step (iii), the solid phase compound is reacted with a boronic acid or ester, or with another organometallic. In step (iv), the protecting group is removed. In step (v), the substituent R_{12′} is introduced. In step (vi), cleavage of the conjugated resin liberates the polycyclic compound of general structure 2. The —OH at the position of R₃ can be converted to another group if desired.

The synthetic procedures of this invention are of general application to a large number of compounds, which differs in the nature, number of substituents of rings A-E and also in the nature of the aromatic rings; being possible its change by polyaromatic rings, heterocyclic rings or polyheterocyclic rings. This versatility together with the facilities of the solid phase approach offers the advantage of its applicability to the synthesis of libraries of compounds and the possibility of a fast production of new compounds for the pharmacological evaluation of a considerable number of new related structures.

Compounds that can be obtained with our methodology are those with the general formula 1 or 2:

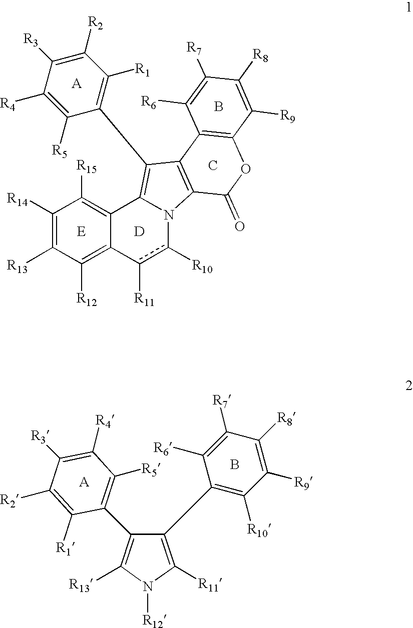

wherein rings A, B and E are preferably aromatic carbocyclic or heterocyclic rings, or with the possible substitution of one or more of this rings for non aromatic heterocyclic rings or for substituted carbocyclic structures;

wherein R₁—R₁₅, R₁′—R₁₁′ and R₁₃′ groups are each independently selected from the group consisting of H, halogen, OH, OR′, SH, SR′, SOR′, SO₂R′, NHR′, N(R′)₂, NHCOR′, N(COR′)₂, NHSO₂R′, OC(═O)H, OC(═O)R′, COOH, COOR′, C₁-C₁₂ alkyl, C₁-C₁₂ haloalkyl, C₂-C₁₂ alkenyl, C₂-C₁₂ alkynyl, substituted or unsubstituted aryl, substituted or unsubstituted aralkyl and substituted or unsubstituted heteroaromatic;

wherein R₁₂′ is selected from the group consisting of H, C₁-C₁₂ alkyl, C₁-C₁₂ haloalkyl, C₂-C₁₂ alkenyl, C₂-C₁₂ alkynyl, substituted or unsubstituted aryl, substituted or unsubstituted arylalkyl and substituted or unsubstituted heteroaromatic;

wherein each of the R′ groups is independently selected from the group consisting of H, OH, NO₂, NH₂, NHalkyl, N(alkyl)₂, SH, Salkyl, CN, halogen, ═O, C(═O)H, C(═O)alkyl, COOH, COOalkyl, substituted or unsubstituted C₁-C₁₈ alkyl, substituted or unsubstituted C₂-C₁₈ alkenyl, substituted or unsubstituted C₂-C₁₈ alkynyl, substituted or unsubstituted aryl; and

wherein the dotted line means an optional double bond.

The lamellarin product can be a natural lamellarin.

Preferably each of R₁ to R₁₅ and R_{1′} to R_{11′} and R_{13′} is chosen from —H, —OH, -halo, —Oalkyl, —OCOalkyl, —COOalkyl, where halo is suitably chloro, and alkyl is suitably methyl. More preferably, at least one of the following is true:

R₁ is H;

R₂ is OH, OMe;

R₃ is OH, OMe;

R₄ is H;

R₅ is H;

R₆ is H;

R₇ is OH, OMe;

R₈ is OH, OMe;

R₉ is H;

R₁₀ is H, OH;

R₁₁ is H;

R₁₂ is H, OH, OMe;

R₁₃ is OH, OMe;

R₁₄ is OH, OMe;

R₁₅ is H.

R₁′ is H;

R₂′ is H;

R₃′ is OH, OMe;

R₄′ is H;

R₅′ is H;

R₆′ is H;

R₇′ is H;

R₈′ is OH, Oalkyl;

R₉′ is H, OH, Oalkyl;

or R₈′ and R₉′ form a fused phenyl ring with the adjacent atoms;

R₁₀′ is H, OH;

R₁₁′ is H, COOalkyl;

R₁₂′ group is H, alkyl especially methyl, or a phenylethyl group of formula:

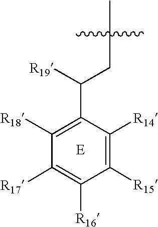

wherein R₁₄′—R₁₈′ groups are each independently selected from the group consisting of H, halogen, OH, OR′, SH, SR′, SOR′, SO₂R′, NHR′, N(R′)₂, NHCOR—, N(COR′)₂, NHSO₂R′, OC(═O)H, OC(═O)R′, COOH, COOR′, C₁-C₁₂ alkyl, C₁-C₁₂ haloalkyl, C₂-C₁₂ alkenyl, C₂-C₁₂ alkynyl, substituted or unsubstituted aryl, substituted or unsubstituted aralkyl and substituted or unsubstituted heteroaromatic; and

wherein R₁₉′ is selected from H, ═O, halogen, OH, OR′, SH, SR′, SOR′, SO₂R′, NHR′, N(R′)₂, NHCOR′, N(COR′)₂, NHSO₂R′, OC(═O)H, OC(═O)R′, COOH, COOR′, C₁-C₁₂ alkyl, C₁-C₁₂ haloalkyl, C₂-C₁₂ alkenyl, C₂-C₁₂ alkynyl, substituted or unsubstituted aryl, substituted or unsubstituted aralkyl and substituted or unsubstituted heteroaromatic; and R₁₃′ is H, COOalkyl.

Each of R₁₄′ to R₁₈′ is preferably chosen from —H, —OH, -halo, —Oalkyl or —OCOalkyl, where halo is suitably bromo, and alkyl is suitably methyl, and R_{19′} is H or ═O. Each can be H. Other suitable definitions include R₁₄′ is OH or OMe; R₁₆′ is OH, OMe or bromo.

The substituents of the aromatic groups R₁—R₉, R₁₂—R₁₅, R₁′—R₁₀′ and R₁₄′—R₁₈′, can also form together with an adjacent substituent an aryl, a cycloalkyl or an heterocyclic group.

Preferred combinations of the various substituents are given in the examples, and may be interchanged.

In the protected compounds having a group PG, the protecting group is not limited to the exemplified groups. Protecting groups for amine and hydroxy functions are extremely well known, and further guidance is unnecessary.

In the compounds of the invention the substituents can be selected in accordance with the following guidance:

Preferred R′ groups are present in groups of formula OR′, COR′ or OCOR′ and include alkyl or alkenyl, that may be substituted at one or more available positions by one or more suitable groups, e.g., halogen such as fluoro, chloro, bromo and iodo, especially w-chloro or perfluoro; aminoalkyl groups such as groups having one or more N atoms and from 1 to about 12 carbon atoms or from 1 to about 6 carbon atoms, and especially including amino acid, notably glycine, alanine, arginine, asparagine, asparaginic acid, cysteine, glutamine, glutamic acid, histidine, isoleucine, leucine, lysine, methionine, phenylalanine, proline, serine, threonine, tryptophan, tyrosine or valine, especially protected forms of such amino acids; carbocylic aryl having 6 or more carbons, particularly phenyl; and aralkyl such as benzyl; heterocyclic groups including heteroalicyclic and heteroaromatic groups, especially with 5 to 10 ring atoms of which 1 to 4 are heteroatoms, more preferably heterocyclic groups with 5 or 6 ring atoms and 1 or 2 heteratoms or with 10 ring atoms and 1 to 3 heteroatoms, the heterocyclic groups optionally being substituted with one or more of the subsitituents permitted for R′ and especially amino such as dimethylamino or with keto.

Suitable halogen substituents in the compounds of the present invention include F, Cl, Br and I.

Alkyl groups preferably have from 1 to 24 carbon atoms. One more preferred class of alkyl groups has 1 to about 12 carbon atoms, yet more preferably 1 to about 8 carbon atoms, still more preferably 1 to about 6 carbon atoms, and most preferably 1, 2, 3 or 4 carbon atoms. Methyl, ethyl and propyl including isopropyl are particularly preferred alkyl groups in the compounds of the present invention. Another more preferred class of alkyl groups has 12 to about 24 carbon atoms, yet more preferably 12 to about 18 carbon atoms, and most preferably 13, 15 or 17 carbon atoms. As used herein, the term alkyl, unless otherwise modified, refers to both cyclic and noncyclic groups, although cyclic groups will comprise at least three carbon ring members.

Preferred alkenyl and alkynyl groups in the compounds of the present invention have one or more unsaturated linkages and from 2 to about 12 carbon atoms, more preferably 2 to about 8 carbon atoms, still more preferably 2 to about 6 carbon atoms, even more preferably 2, 3 or 4 carbon atoms. The terms alkenyl and alkynyl as used herein refer to both cyclic and noncyclic groups, although straight or branched noncyclic groups are generally more preferred.

Preferred alkoxy groups in the compounds of the present invention include groups having one or more oxygen linkages and from 1 to about 12 carbon atoms, more preferably from 1 to about 8 carbon atoms, and still more preferably 1 to about 6 carbon atoms, and most preferably 1, 2, 3 or 4 carbon atoms.

Preferred alkylthio groups in the compounds of the present invention have one or more thioether linkages and from 1 to about 12 carbon atoms, more preferably from 1 to about 8 carbon atoms, and still more preferably 1 to about 6 carbon atoms. Alkylthio groups having 1, 2, 3 or 4 carbon atoms are particularly preferred.

Preferred alkylsulfinyl groups in the compounds of the present invention include those groups having one or more sulfoxide (SO) groups and from 1 to about 12 carbon atoms, more preferably from 1 to about 8 carbon atoms, and still more preferebly 1 to about 6 carbon atoms. Alkylsulfinyl groups having 1, 2, 3 or 4 carbon atoms are particularly preferred.

Preferred alkylsulfonyl groups in the compounds of the present invention include those groups having one or more sulfonyl (SO₂) groups and from 1 to about 12 carbon atoms, more preferably from 1 to about 8 carbon atoms, and still more preferably 1 to about 6 carbon atoms. Alkylsulfonyl groups having 1, 2, 3 or 4 carbon atoms are particularly preferred.

Preferred aminoalkyl groups include those groups having one or more primary, secondary and/or tertiary amine groups, and from 1 to about 12 carbon atoms, more preferably 1 to about 8 carbon atoms, still more preferably 1 to about 6 carbon atoms, even more preferably 1, 2, 3 or 4 carbon atoms. Secondary and tertiary amine groups are generally more preferred than primary amine moieties.

Suitable heterocyclic groups include heteroaromatic and heteroalicyclic groups. Suitable heteroaromatic groups in the compounds of the present invention contain one, two or three heteroatoms selected from N, O or S atoms and include, e.g., coumarinyl including 8-coumarinyl, quinolinyl including 8-quinolinyl, pyridyl, pyrazinyl, pyrimidyl, furyl, pyrrolyl, thienyl, thiazolyl, oxazolyl, imidazolyl, indolyl, benzofuranyl and benzothiazol. Suitable heteroalicyclic groups in the compounds of the present invention contain one, two or three heteroatoms selected from N, O or S atoms and include, e.g., tetrahydrofuranyl, tetrahydropyranyl, piperidinyl, morpholino and pyrrolindinyl groups.

Suitable carbocyclic aryl groups in the compounds of the present invention include single and multiple ring compounds, including multiple ring compounds that contain separate and/or fused aryl groups. Typical carbocyclic aryl groups contain 1 to 3 separate or fused rings and from 6 to about 18 carbon ring atoms. Specifically preferred carbocyclic aryl groups include phenyl including substituted phenyl such as 2-substituted phenyl, 3-substituted phenyl, 2,3-substituted phenyl, 2,5-substituted phenyl, 2,3,5-substituted and 2,4,5-substituted phenyl, including where one or more of the phenyl substituents is an electron-withdrawing group such as halogen, cyano, nitro, alkanoyl, sulfinyl, sulfonyl and the like; naphthyl including 1-naphthyl and 2-naphthyl; biphenyl; phenanthryl; and anthracyl.

References herein to substituted R′ groups in the compounds of the present invention refer to the specified moiety, typically alkyl or alkenyl, that may be substituted at one or more available positions by one or more suitable groups, e.g., halogen; cyano; hydroxyl; nitro; azido; C₁-C₆ alkanoyl; carboxamido; C₁-C₁₂ alkyl; C₂-C₁₂ alkenyl and alkynyl; C₁-C₁₂ alkoxy; aryloxy such as phenoxy; C₁-C₁₂ alkylthio groups including those moieties having one or more thioether linkages; C₁-C₁₂ alkylsulfinyl groups including those moieties having one or more sulfinyl linkages; C₁-C₁₂ alkylsulfonyl groups including those moieties having one or more sulfonyl linkages; C₁-C₁₂ aminoalkyl groups such as groups having one or more N atoms; carbocylic aryl having 6 or more carbons, particularly phenyl; aralkyl such as benzyl; heterocyclic groups including heteroalicyclic and heteroaromatic groups, especially with 5 to 10 ring atoms of which 1 to 4 are heteroatoms, more preferably heterocyclic groups with 5 or 6 ring atoms and 1 or 2 heteratoms or with 10 ring atoms and 1 to 3 heteroatoms.

The solid phase synthesis of compounds of structures 1 and 2 has been done using as a solid support Merrifield or Wang resins. It is also possible to use other resins (The Combinatorial Index, B. A. Bunin, Academic Press 1998) with adequate functionalization. Both resins used for preparation of 1 and 2 have given good results in the synthetic process. Furthermore, the modulation of the cleavage conditions can afford different substitution of the polycyclic compound.

Schemes 1 and 2 shown the preferred process for the synthetic formation of the target compounds. In scheme 1 the OH group of the halophenol is fixed in m-position and in scheme 2 is fixed in p-position, but these fixed structures should not be interpreted as limitative. The halophenol compound can present the hydroxyl group in any available position.

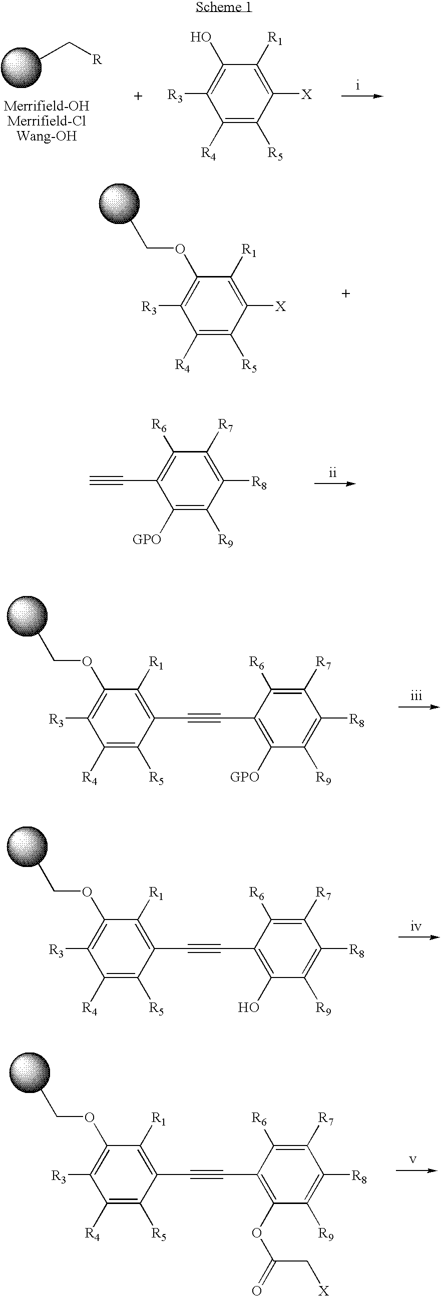

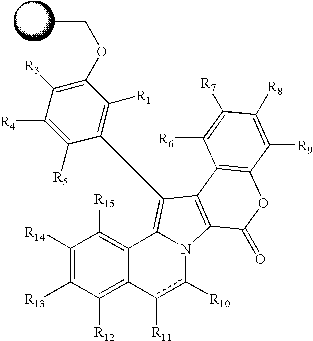

This process comprises the following sequential key steps:

i) Substituted halophenols, preferably iodophenols, were attached to the Merrifield or Wang resins forming a ether bond, as it is described in Examples 1-3, and 27-29 to give the attached phenols of structure I.

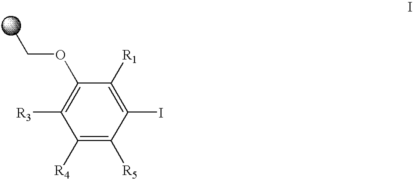

IR spectroscopy was used for monitoring solid phase reaction (B. Yan, G. Kumaravel, H. Anjaria, A. Wu, R. C. Petter, C. F. Jewell, Jr., and J. R. Wareing, J. Org. Chem., 1995, 60, 5736; B. Yan, Acc. Chem. Res., 1998, 31, 621) and indicates the end of the reaction for the disappearance of the OH stretch vibration at 3450 and 3580 cm⁻¹ characteristic of the hydroxyl group of the resin as well as by Carbon Magic Angle Spin Nuclear Magnetic Resonance (¹³C MAS-NMR).

The scope of attachment of iodophenol reactions was tested after cleavage, ¹H NMR and HPLC were used to analyse the purity of cleaved phenols. Merrifield conjugated iodophenols I were cleaved using a Lewis acid, such as: a) ZnBr₂ and AcBr, (W-R. Li, Y-C. Yo, Tetrahedron Lett., 1999, 40, 9085) to give 5-Iodo-2-methoxyphenyl acetate (Example 5) and 4-Iodophenyl acetate (Example 6), b) SnCl₄ affording the 5-Iodo-2-methoxyphenol (Example 4) or c) AlCl₃ (E. G. Mata, Tetrahedron Lett., 1997, 38, 6335). For the Wang resin a protic acid-media such as TFA in DCM (B. C. Hamper, D. R. Dukesherer, M. S. South, Tetrahedron Lett., 1996, 37, 3671) was used for the cleavage to give 6-Iodo-2-methoxyphenol (Example 7). This procedure requires shorter reaction time than the two previously described procedures.

ii) The bisarylacetylene compounds of structure II were obtained by coupling reaction, catalyzed by Palladium, of the anchored iodophenol I with an arylacetylene of structure III (see for example, K. Sonogashira, Handbook of Organopalladium Chemistry for Organic Synthesis, 2002, 1, 493, Willey).

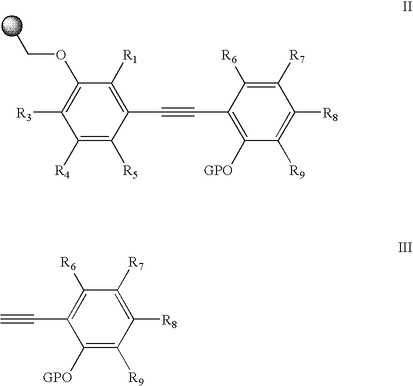

The aryl acetylene III was prepared in solution by standard literature procedures (Comprehensive Organic Synthesis, Trost-Fleming, Vol. 3, 521 and 551, Pergamon).

iii) The substituent at position 2 of III has to be a protected hydroxy group (GPO=MOM) or a synthetically precursor of the hydroxy group (GPO═CHO): Protective Groups in Organic Synthesis, T. W. Greene, P. G. M. Wuts, Wiley Intercience.

When GPO was a formyl group its transformation in phenol was done using Baeyer-Villiger conditions (Comprehensive Organic Synthesis, Trost-Fleming, Vol. 7, 671 Pergamon) followed by hydrolysis of the generated formate. The formyl resin II (GPO═CHO) was treated with m-CPBA and NaHCO₃ in DCM at room temperature. Formation of the formate was indicated by the strong IR absorption at 1730 cm⁻¹. Hydrolysis of formate using KOH or NaOH in MeOH-THF afforded the phenol resin II (GPO═OH). Absorption at 3417 cm⁻¹ and the disappearance of absorption at 1730 cm⁻¹ in the IR are the most important spectroscopic data for showing the transformation.

On the other hand, elimination of O-methoxymethyl (MOM) protecting group with trimethylsilyl bromide (Exemple 38) afforded the phenol. Its formation was tested by IR spectroscopy.

iv) The haloacetate IV, preferably iodoacetate, was prepared by reaction of II (GPO═OH) with iodo, bromo or chloro acetic acid in the standard conditions of ester formation with a polar solvent, like DMF, using an activating agent, as such an example DIPAD or DCC, and a base, as DMAP, Examples 20-21, and 39. ¹³C MAS-NMR and IR were used to test the ester formation on solid phase.

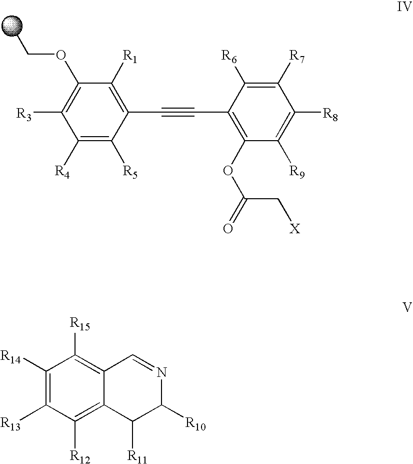

v) Alkylation of IV with a 3,4-dihydroisoquinoline V followed by a dipolar (2+3) cycloaddition afforded the pentacyclic system characteristic of 1, Examples 22-23, and 40. The substituted isoquinolines V were prepared in solution by described procedures (Heterocyclic compounds. Isoquinolines, Coppola-Schuster, Vol 38, Part. 3, 266. Interscience, 1995) starting from available benzaldehydes or phenethylamines. A simultaneous formation of pyrrole and lactone rings was produced during the dipolar cycloaddition. IR spectra of resins IV and 1 showed the absorption of esther and lactone groups, as well as, the absence of the triple bond absorption in 1. Oxidation procedure, such as for example with DDQ, afforded the C—C double bond present in some natural lamellarins.

Cleavage reaction of the conjugated 1 Merrifield resin (1-MR) to liberate the polycyclic compound of general structure 1 was done by using a Lewis acid, such as ZnBr₂ in AcBr or AlCl₃ in DCM. On the other hand, cleavage reaction of the conjugated Wang resin (1-WR) was done by using a protic acid media, such as TFA in DCM.

When the conjugate MR has R₁═R₄═R₅═R₆═R₉═R₁₀═R₁₁═R₁₂═R₁₅═H, R₃═R₇═R₁₃═R₁₄═OMe, and R₈═OiPr (9-MR) cleavage with ZnBr₂ afforded the 3,3′-di-O-acetyllamellarin U (Example 24) and with AlCl₃ in DCM lamellarins U and L were obtained (Example 25). The same conjugated Wang resin cleaved with TFA yielded 3-O-Isopropyllamellarin U (Example 26).

When the conjugate MR has R₁═R₄═R₅═R₆═R₈═R₉═R₁₀═R₁₁═R₁₂═R₁₅═R₃═R₁₃═R₁₄═OMe, R₇═F, and R₈═OiPr (31-MR) cleavage with AlCl₃ in DCM lamellarin 32 was obtained (Example 41).

When the conjugate MR has R₁═R₄═R₅═R₆═R₉═R₁₀═R₁₁═R₁₂═R₁₅═H, R₃═R₇═R₁₃═R₁₄═OMe, R₈═OiPr and C7-C8 is a double bond (45-MR) cleavage with ZnBr₂ afforded the 3,3′-Di-O-acetyl-11-methoxylamellarin N (Example 58) and with AlCl₃ in DCM lamellarin N and 10-Methoxylamellarin N were obtained (Example 59).

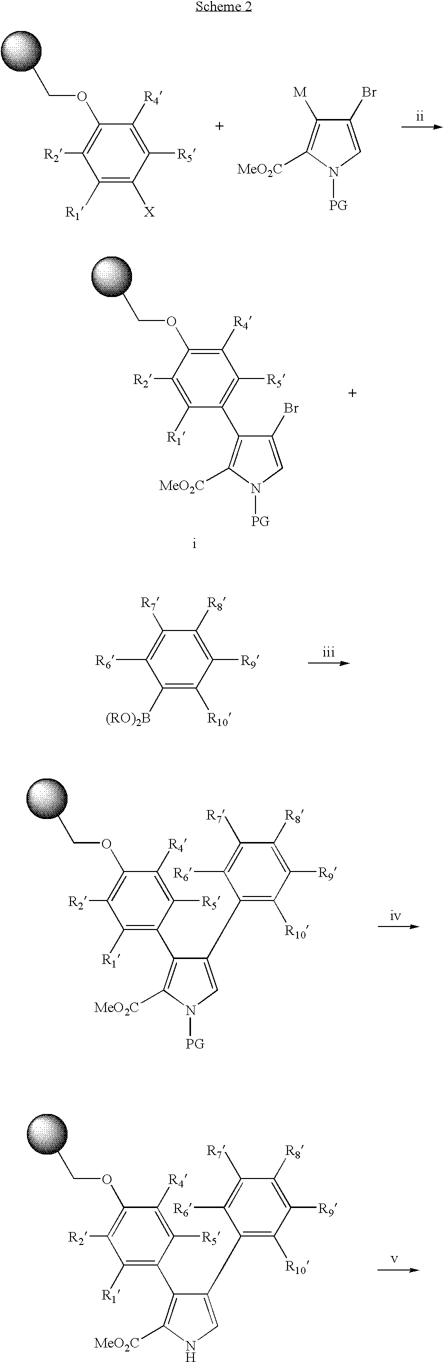

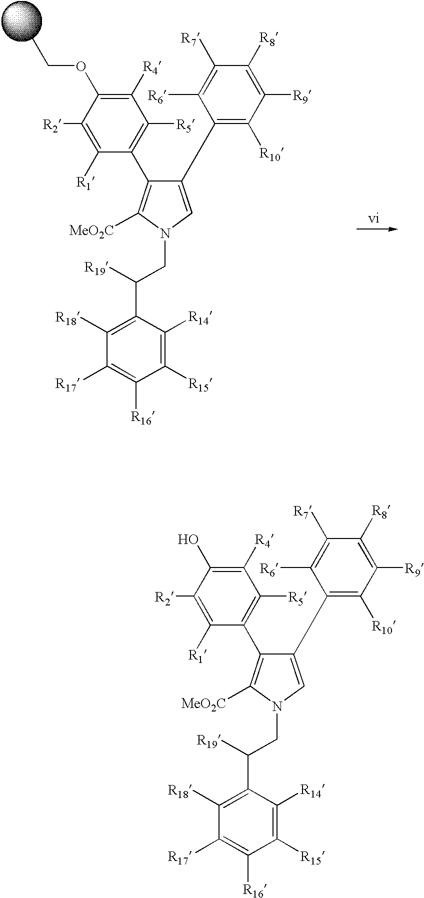

The process of Scheme 2 comprises the following sequential key steps:

The solid phase synthesis of compounds of structure 2 was done using a Merrifield resin. The attachment of iodophenols to the Merrifield-Cl (W-R. Li, Y-C.- Yo, Tetrahedron Lett., 1999, 40,-9085) resin was done in basic conditions.

i) The methyl 3,4-dibromo-1-(triisopropylsilyl)-1H-pyrrole-2-carboxylate VI (B. L. Bray, P. H. Mathies, R. Naef, D. R. Solas, T. T. Tidwell, D. R. Artis, J. M. Muchoswski, J. Org. Chem., 1990, 55, 6317) was used in the preparation of 2 as a building block. Selective ortho-directed interchange of bromine or chloride in position 3 by lithium was produced after treatment of VI with n-BuLi, LDA or t-BuLi at low temperature. Transmetalation with a) zinc (ZnCl₂ or ZnBr₂; Negishi conditions (Palladium in Heterocyclic Chemistry, J. J. Li, G. W. Gribble Tetrahedron Organic Chemistry Series, Vol. 20, 43, Pergamon)), b) tin (n-Bu₃SnCl or Me₃SnCl; Stille conditions (J. K. Stille et al J. Am. Chem. Soc., 1984, 106, 4630)) or c) borane ((MeO)₃B; Suzuki conditions (Handbook of Organopalladium Chemistry for Organic Synthesis, 2002, 1, 493, Willey)) gave the organometallic compound used for cross coupling reaction ii) with the resin-attached phenol I. This reaction ii) is catalyzed by Palladium. The compound of general structure VII was prepared following that general procedure. VII contains the first diversity group.

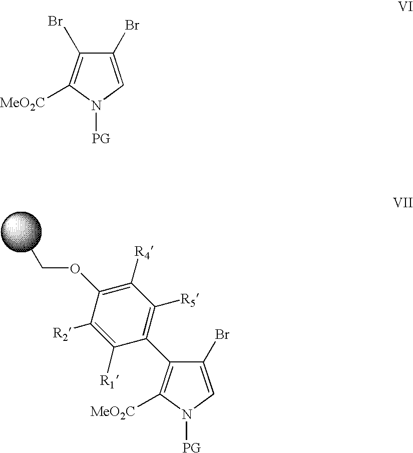

The formation of VII could be tested by ¹³C MAS-NMR as in Example 10 or by cleavage reaction and characterization of the resulting product as it is described in Example 11.

iii) A new Palladium catalyzed coupling reaction between VII and a boronic ester or acid under Suzuki conditions (Handbook of Organopalladium Chemistry for Organic Synthesis, 2002, 1, 493, Willey), (examples 12,42-44) introduces the second diversity element giving a substituted pyrrolecarboxylate of general structure VIII. As well as boronic ester or acid, other organometallic could be used for this coupling reaction.

N-Deprotection (iv) followed by alkylation (v) introduces the last diversity element over the compound of general structure IX.

iv) In the example using triisopropylsilyl as protecting group, the solid phase N-desilylation was done using NH₄F in DCM and methanol giving the anchored diarylpyrrole of structure VIII (R═H). The Gel Phase ¹³C NMR of VIII (R═H) clearly indicates the absence of methyl signal proper of triisopropylsilyl substituent. Cleavage of VIII with AlCl₃ in DCM gave lamellarin Q (R═H, Example 45).

v) N-alkylation of VIII (PG=R₁′═R₂′═R₄′═R₅′═R₆′═R₇′═R₉′═R₁₀′═H, R₈′═OMe) with the 2-bromo-1-(4-methoxyphenyl)ethanone 16 was done using procedures described in the literature such as: NaH or LDA in dry THF giving IX (R₁′═R₂′═R₄′═R₅′═R₆′═R₇′═R₉═R₁₀′═R₁₄′═R₁₅′═R₁₇′═R₁₈′═H, R₁₉′═ ═O, R₈′═R₁₆′═OMe). N-alkylation of VIII (PG=R₁′═R₂′═R₄′═R₅′═R₆′═R₇′═R₉′═R₁₀′═H, R₈′═OiPr) with the 2-bromo-1-(4-methoxyphenyl)ethanone 16 was done using K₂CO₃, and 18-crown-6 in DMF to afford IX (R₁′═R₂′═R₄′═R₅′═R₆′═R₇′═R₉′═R₁₀′═R₁₄′═R₁₅′═R₁₇′═R₁₈′═H, R₁₉′═ ═O, R₈′═OiPr, R₁₆′═OMe) and when 2-bromophenethyl 4-methylbenzenesulfonate 41 was used IX (R₁′═R₂′═R₄′═R₅′═R₆′═R₇′═R₉′═R₁₀′═R₁₉′═R₁₅′═R₁₇′═R₁₈′═H, R₈═OiPr, R₁₄′═Br) was obtained. N-alkylation of VIII (PG=R₁′═R₂′═R₄′═R₅′═R₆′═R₇′═R₉′═R₁₀′═H, R₈′═OiPr) with methyl iodide was done using K₂CO₃, and 18-crown-6 in DMF in a microwave oven to afford VII (PG=Me, R₁′═R₂′═R₄′═R₅′═R₆′R₇′═R₉′═R₁₀′═H, and R₈′═OiPr).

vi) Cleavage of IX with AlCl₃ in DCM gave lamellarin O (Example 56), and Dimethoxylamellarin O (Example 15).

Cleavage of IX with SnCl₄ in DCM gave compound 35 (Example 46).

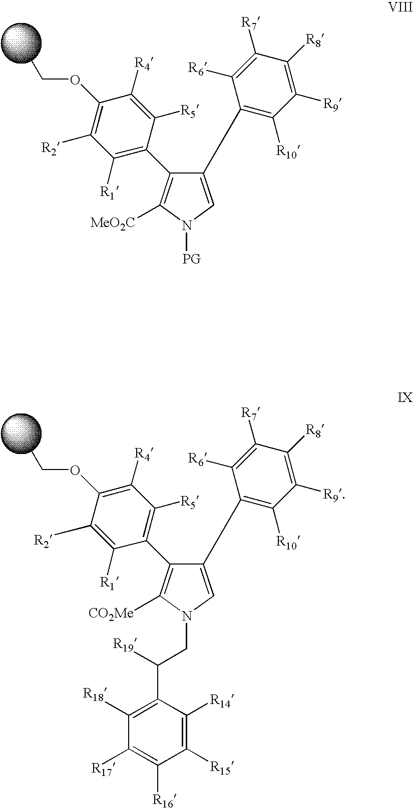

Using these processes compounds of formula 1 and 2 as defined above can be prepared.

The following examples are illustrative of the present invention and should not be interpreted as limitative.

### EXAMPLES

General Procedures. Wang resin and Merrifield resin were from ABI (Framingham, Mass.), Bachem (Bubendorf, Switzerland), NovaBiochem (Läufelfingen, Switzerland).

DIPEA, DIPCDI, and TFA were purchased from Aldrich (Milwaukee, Wis.). DMF and DCM were purchased from SDS (Peypin, France). Acetonitrile (HPLC grade) was purchased from Scharlau (Barcelona, Spain). All commercial reagents and solvents were used as received with exception of DCM, which was passed through an alumina column to remove acidic contaminants.

Solid-phase syntheses were carried out in polypropylene syringes (10-50 mL) fitted with a polyethylene porous disc. Solvents and soluble reagents were removed by suction. Syntheses carried out on solid-phase were controlled by RP-HPLC of the intermediates obtained after cleaving of the resin. HPLC reversed phase Symmetry columns C₁₈ 4,6×150 mm, 5 μm was from Waters (Ireland). Analytical HPLC was carried out on a Waters instrument 996 photodiode array detector equipped with the Waters 2695 separation module and the millennium software.

APCI and ES-MS analysis of samples were performed in a PerSeptive Biosystems Voyager DE RP, using DHB matrix, and in a Waters Micromass ZQ spectrometer. ¹H-NMR spectroscopy at 278° K was performed on a Varian Unity Plus (500 MHz). Chemical shifts (δ) are expressed in parts per million downfield from TMS. Coupling constants (J) are expressed in hertz.

The following additional abbreviations are used: DIPEA, N,N-diisopropylethylamine; DMF, N,N-dimethylformamide; DCM, dichloromethane; HPLC, high-performance liquid chromatography; MS, mass spectrometry; RP, reverse phase; TFA, trifluoroacetic acid. All solvent ratios are volume/volume unless stated otherwise.

### Example 1

### 5-Iodo-2-methoxyphenoxy-resin 3-MR

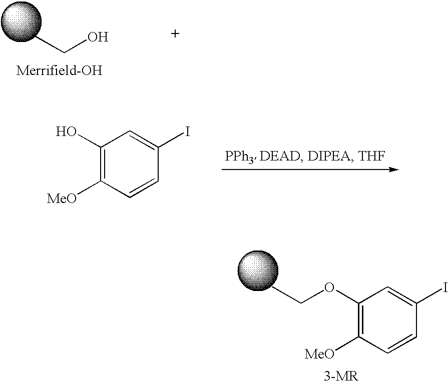

Merrifield-OH resin (1.0 g of 100-200 mesh material, 0.68 mmol/g loading) was washed with DCM and THF (1×10 mL, each) and dried. To the swelled resin with THF (15 mL), 2-methoxy-5-iodophenol (510 mg, 2.04 mmol, 3 equiv.), PPh₃ (535 mg, 2.04 mmol, 3 equiv.), DIPEA (1.05 mL, 6.12 mmol, 9 equiv.) were added and the resulting mixture was shaken at 0° C. for 10 min. DEAD (320 μL, 2.04 mmol, 3 equiv.) was added dropwise at the same temperature and the resulting mixture was shaken at room temperature for 3 h. The solvent was then removed. The resin was washed with DMF, DCM, MeOH, and Et₂O (5×15 mL each). The resin was dried under reduced pressure. ¹³C MAS-NMR(125 MHz): δ 149.8, 149.2, 130.1, 122.7, 113.7, 82.2 (C5), 55.9 (OCH₃).

### Example 2

### 5-Iodo-2-methoxyphenoxy-resin 3-WR

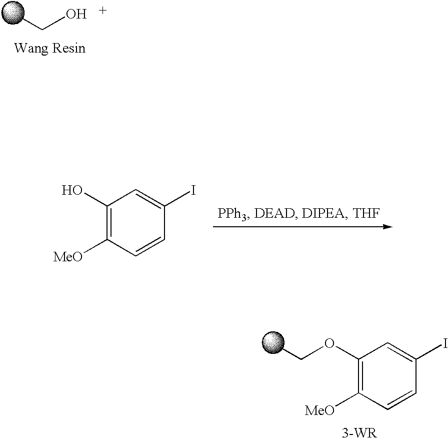

Wang resin (1.0 g, 0.86 mmol/g loading) was washed with DCM and THF (1×10 mL, each) and dried. To the swelled resin with THF (15 mL), 2-methoxy-5-iodophenol (645 mg, 2.58 mmol, 3 equiv.), PPh₃ (680 mg, 2.58 mmol, 3 equiv.), DIPEA (1.32 mL, 7.74 mmol, 9 equiv.) were added and the resulting mixture was shaken at 0° C. for 10 min DEAD (410 μL, 2.58 mmol, 3 equiv.) was added dropwise at the same temperature and the resulting mixture was shaken at room temperature for 3 h. The solvent was then removed. The resin was washed with DMF, DCM, MeOH, and Et₂O (5×15 mL, each) and was dried under reduced pressure. δ 149.8, 149.2, 130.1, 122.7, 113.7, 82.2 (C5), 55.9 (OCH₃).

### Example 3

### 4-Iodophenoxy-resin 10-MR

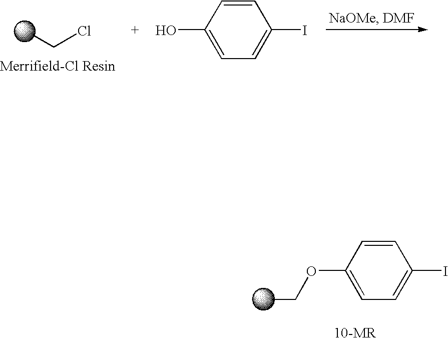

Merrifield-Cl resin (0.5 g, 0.61 mmol/g loading) was swelled in DMF (15 mL) for 30 min. and then 4-iodophenol (268 mg, 1.22 mmol, 5 equiv.) and NaOMe (280 μL, 1.22 mmol, 4.4 M) in DMF were added. The reaction mixture was warmed at 80° C. and shaken in a vibromatic during 24 h. After this time the resin was washed with DCM, DMF:DCM (1:1), DMF, DCM, MeOH, Et₂O (3×3 mL, each) and was dried at vacuum. Cleavage of a sample with the experimental conditions described in Example 6 showed a quantitative yield of attachment reaction. IR (KBr) 1872, 1803, 1599, 743.

### Example 4

### 5-Iodo-2-methoxyphenol (cleavage with SnCl₄)

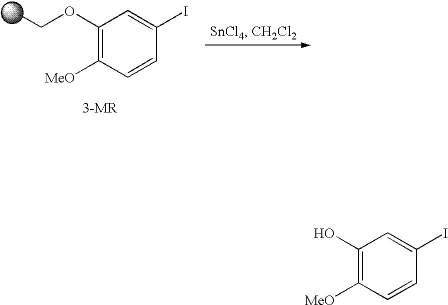

The Merrifield resin 3-MR (100 mg) was swelled with dry DCM for 30 min, SnCl₄ (124 μL, 0.68 mmol, 10 equiv.) was added and the reaction mixture was shaken under Ar overnight. After this time the resin was filtered off and washed with DCM (5×5 mL). The filtrates were washed with H₂O (6×15 mL), dried (MgSO₄ anh.), and concentrated under reduce pressure to give 5-iodo-2-methoxyphenol (16 mg of crude).

¹H NMR (200 MHz, CDCl₃) δ 7.23 (d, J=2.2 Hz, 1H, H-6), 7.15 (dd, J=8.4 and 2.2 Hz, 1H, H-4), 6.59 (d, J=8.4 Hz, 1H, H-3), 5.60 (bs, 1H, OH), 3.87 (s, 3H, OCH₃).

¹³C NMR (75 MHz, CDCl₃) δ 146.6, 146.5, 129.0 (d, C-3), 123.4(d, C-4), 112.4 (d, C-6), 83.0 (s, C-5), 56.0 (q, OCH₃).

### Example 5

### 5-Iodo-2-methoxyphenyl acetate (cleavage with ZnBr₂/AcBr)

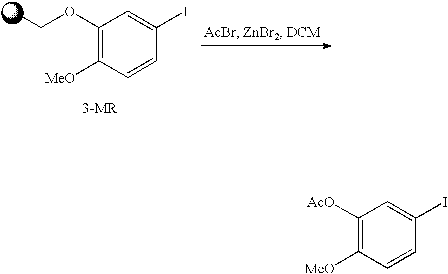

The Merrifield resin 3-MR (100 mg) was swelled for 30 minutes with dry DCM (1 mL). After this time anh. ZnBr₂ (60.1 mg, 0.17 mmol, 4 equiv.) and AcBr (50 μL, 0.68 mmol, 10 equiv.) were added and the reaction mixture was then shaken under Ar overnight at room temperature. The resin was filtered off and washed with DCM (5×5 mL). The filtrates were washed with aq. 5% NaHCO₃ (5×15 mL), 2 M HCl (3×15 mL) and brine (3×15 mL). The organic solution was dried (MgSO₄ anh.), filtered and concentrated under reduce pressure to give 5-iodo-2-methoxyphenyl acetate (13 mg, 83%).

¹H NMR (200 MHz, CDCl₃) δ 7.49 (dd, J=8.8 and 2.0 Hz, 1H, H-4), 7.33 (d, J=2.0 Hz, 1H, H-6), 6.72 (d, J=8.8 Hz, 1H, H-3), 3.81 (bs, 3H, OCH₃), 2.30 (s, 3H, COCH₃).

¹³C NMR (50 MHz, CDCl₃) δ 168.5 (s, C═O), 151.3 (s, C-2), 140.4 (s, C-1), 135.6 (d, C-4), 131.5 (d, C-6), 114.2 (d, C-3), 81.3 (s, C-5), 56.0 (q, OCH₃), 20.6 (q, COCH₃).

### Example 6

### 4-Iodophenyl acetate (cleavage with ZnBr₂ and AcBr)

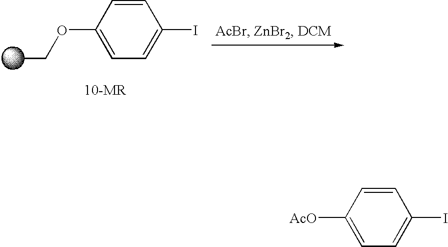

The resin 10-MR (40 mg, 0.021 mmols, 0.52 mmol/g) was swelled in dry DCM (2 mL) under N₂ and ZnBr₂ (16.4 mg, 0.073 mmol, 3.5 equiv.) and AcBr (62 μL, 0.832 mmol, 40 equiv.) were added. The reaction mixture was shaken in the vibromatic for 24 h at room temperature. After this time was filtered and washed with DCM. The organic solution was washed with aq. 5% NaHCO₃, aq. 5% HCl, saturated NaCl, dried (Na₂SO₄ anh.) and evaporated (2.5 mg of crude, 50%).

¹H NMR (200 MHz, CDCl₃) δ 2.29 (s, 3H, CH₃), 6.86 (d, J=8.8 Hz, 2H, H-2 and H-6), 7.68 (d, J=8.8 Hz, 2H, H-3 and H-5).

### Example 7

### 5-Iodo-2-methoxyphenol (cleavage with TFA:DCM)

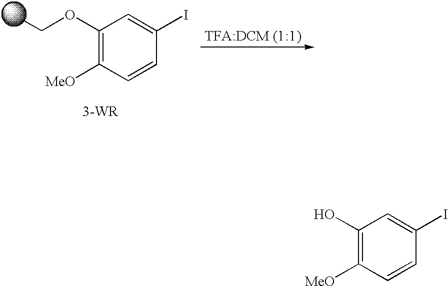

A solution of TFA in DCM (1:1, 2 mL) was added-to the Wang resin 3-WR (56 mg, 0.71 mmol/g loading) and the mixture was shaken for 2 h at room temperature. The resulting suspension was filtered off, the same acid solution was added and the mixture was shaken for 2 h. This process was repeated twice. Finally, the resin was washed several times with DCM. The filtrates were washed with H₂O (3×25 mL), dried (MgSO₄ anh.), and concentrated under reduced pressure to give a very clean 5-iodo-2-methoxyphenol as showed by HPLC.

¹H NMR (400 MHz, CDCl₃) δ 7.23 (d, J=2.2 Hz, 1H, H-6), 7.15 (dd, J=8.4 and 2.2 Hz, 1H, H-4), 6.59 (d, J=8.4 Hz, 1H, H-3), 5.60 (bs, 1H, OH), 3.87 (s, 3H, OCH₃).

### Example 8

### 5-(2-formyl-4-isopropoxy-5-methoxy-phenylethynyl)-2-methoxy-phenoxy-resin 5-MR

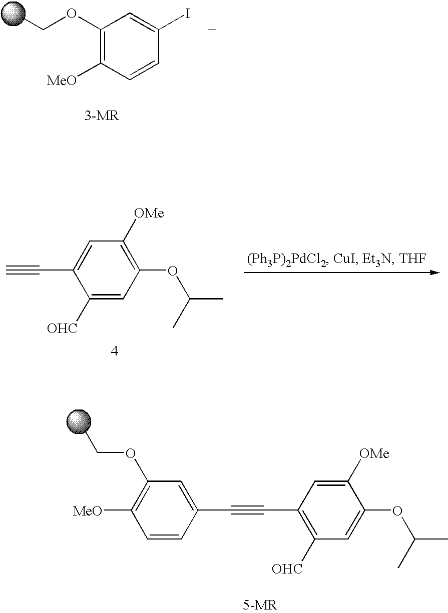

A solution of 2-ethynyl-5-isopropoxy-4-methoxy-benzaldehyde 4 (221 mg, 1.01-mmol, 4 equiv.) in THF and CuI (30 mg, 0.08 mmol, 0.6 equiv.) was added to Merrifield resin 3-MR (506 mg, 0.5 mmol/g loading) swelled with a mixture of THF:DIPEA (3:1) for 30 min. The resulting suspension was bubbled with N₂ for 10 min and (Ph₃P)₂PdCl₂ (53 mg, 0.08 mmol, 0.3 equiv.) was added and the reaction mixture was shaken at room temperature for 20 h. The resin was filtered and washed with THF, DMF, 0.02 M sodium dihexyldithiocarbamate in DMF, DMF, THF, DCM, MeOH (5×15 mL, each) and dried under reduce pressure.

IR (KBr, cm⁻¹) v 2200 (C≡C), 1682 (HC═O).

¹H MAS-NMR (500 MHz, CDCl₃) δ 10.62 (HC═O), 3.96 (2 OCH₃), 1.38 (CH(CH₃)₂).

¹³C MAS-NMR (125 MHz) δ 190.5 (HC═O), Arom. (154.6, 147.9, 132.0, 131.9, 131.9, 129.8, 121.4, 114.5, 110.9), 95.0 (Ar—C≡C—Ar), 83.6 (Ar—C≡C—Ar), 71.2 (OCH(CH₃)₂), 56.1 (OCH₃), 55.8 (OCH₃), 21.8 (CH(CH₃)₂).

### Example 9

### 5-(2-formyl-4-isopropoxy-5-methoxy-phenylethynyl)-2-methoxy-phenoxy-resin 5-WR

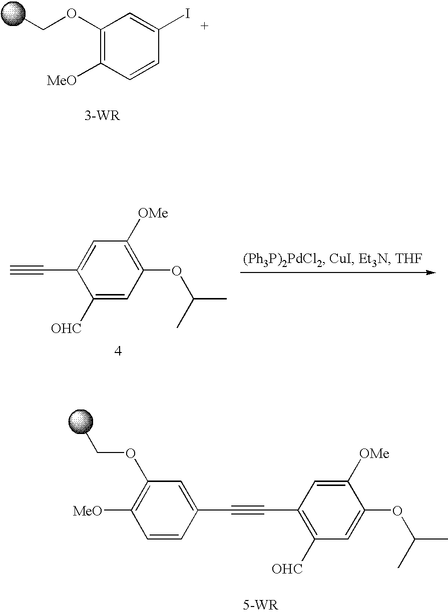

A solution of 2-ethynyl-5-isopropoxy-4-methoxy-benzaldehyde 4 (0.56 g, 2.58 mmol, 3 equiv.) in THF and CuI (0.1 g, 0.6 mmol, 0.6 equiv.) was added to Wang resin 3-WR (1.087 g, 0.71 mmol/g loading) swelled with a mixture of THF:DIPEA (3:1) for 30 min. The resulting suspension was bubbled with N₂ for 10 min and (Ph₃P)₂PdCl₂ (0.18 g, 0.26 mmol, 0.3 equiv.) was added and the reaction mixture was shaken at room temperature for 20 h. The resin was filtered and washed with THF, DMF, 0.02 M sodium dihexyldithiocarbamate in DMF, DMF, THF, DCM, MeOH (5×15 mL, each) and dried under reduce pressure.

IR (KBr, cm⁻¹) v 2200 (C≡C), 1682 (HC═O).

¹H MAS-NMR (500 MHz, CDCl₃) δ 10.62 (HC═O), 3.96 (2 OCH₃), 1.38 (CH(CH₃)₂).

¹³C MAS-NMR (125 MHz) δ 190.5 (HC═O), Arom. (154.6, 147.9, 132.0, 131.9, 131.9, 129.8, 121.4, 114.5, 110.9), 95.0 (Ar—C≡C—Ar), 83.6 (Ar—C≡C—Ar), 71.2 (OCH(CH₃)₂), 56.1 (OCH₃), 55.8 (OCH₃), 21.8 (CH(CH₃)₂).

### Example 10

### 4-(4-Bromo-2-methoxycarbonyl-1-(triisopropylsilyl)-1H-pyrrol-3-yl)-phenoxy-resin 12a-MR

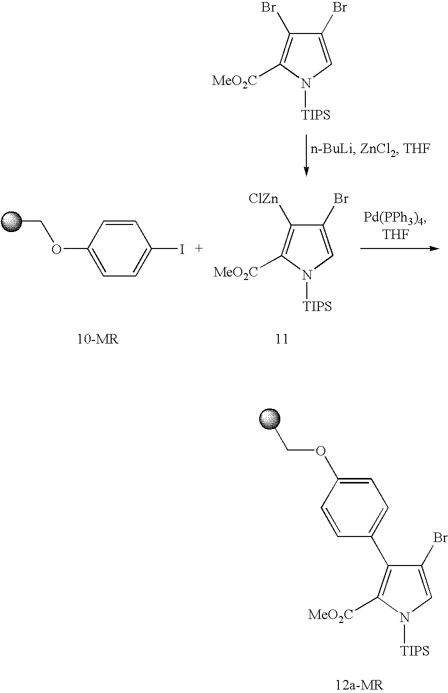

n-BuLi (11.72 mL, 18.75 mmol, 1.6 M) was added dropwise to a cooled (−78° C.) solution of methyl 3,4-dibromo-1-(triisopropylsilyl)-1H-pyrrole-2-carboxylate (8.23 g, 18.75 mmol) in dry THF (54 mL) under N₂ and the mixture was stirred for 15 min at that temperature. After this time a solution of ZnCl₂ (4.09 g, 30,01 mmol) in dry THF (8 mL) was added and the reaction mixture was stirred for 5 min at −78° C. and 25 min at room temperature. The reaction mixture was added with a transfer to the resin 10-MR (3.61 g, 1.87 mmol, 0.52 mmol/g) swelled with THF (15 mL) and Pd(PPh₃)₄ (325 mg, 0.28 mmol, 0.15 equiv.) using N₂. The reaction mixture was stirred 24 h at room temperature. After this time the resin was washed with THF, DCM, MeOH, Et₂O and was dried in the vacuum oven at 40° C.

¹³C MAS-NMR (125 MHz, CDCl₃) δ 13.4 (CH(CH₃)₂), 18.3 (CHCH₃), 50.9 (CO₂CH₃).

IR (KBr) 1697, 1600, 741.

### Example 11

### Methyl 3-(4-acetoxyphenyl)-5-acetyl-4-bromo-1H-pyrrole-2-carboxylate 12b

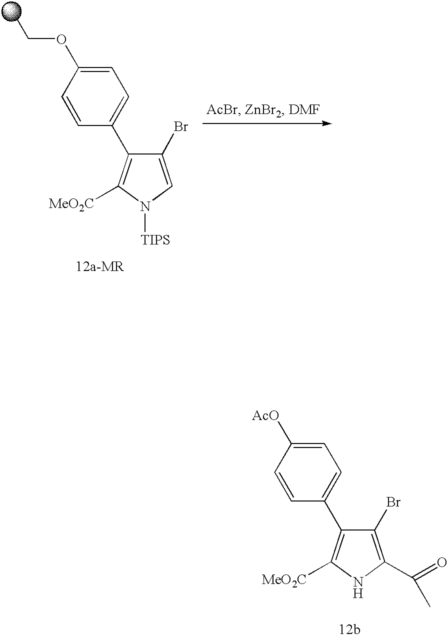

The methyl 3-(4-acetoxyphenyl)-5-acetyl-4-bromo-1-(triisopropylsilyl)-1H-pyrrole-2-carboxylate 12b was obtained from 12a-MR following the cleavage conditions described in the Example 6.

¹H NMR (200 MHz, CDCl₃) δ 2.33 (s, 3H, COCH₃), 2.72 (s, 3H, OCOCH₃), 3.75 (s, 3H, CO₂CH₃), 7.17 (d, J=8.7 Hz, 2H, H-3, H-5), 7.35 (d, J=8.7 Hz, 2H, H-2, H-6).

¹³C NMR (CDCl₃, 50 MHz) δ 17.7 (q, CH₃), 21.3 (q, CH₃), 52.2 (q, CH₃), 104.9 (s), 120.8 (d), 122.2 (s), 129.4 (s), 130.6 (s), 130.9 (s), 131.6 (d), 150.4 (s), 165.1 (s), 169.2 (s), 188.2 (s).

MS (EI) 381 (⁸¹BrM⁺, 11), 379 (⁷⁹BrM⁺, 11), 337 (74), 339 (72).

### Example 12

### 4-(2-Methoxycarbonyl-4-(4-methoxyphenyl)-1-(triisopropylsilyl)-1H-pyrrol-3-yl)-phenoxy 14a-MR

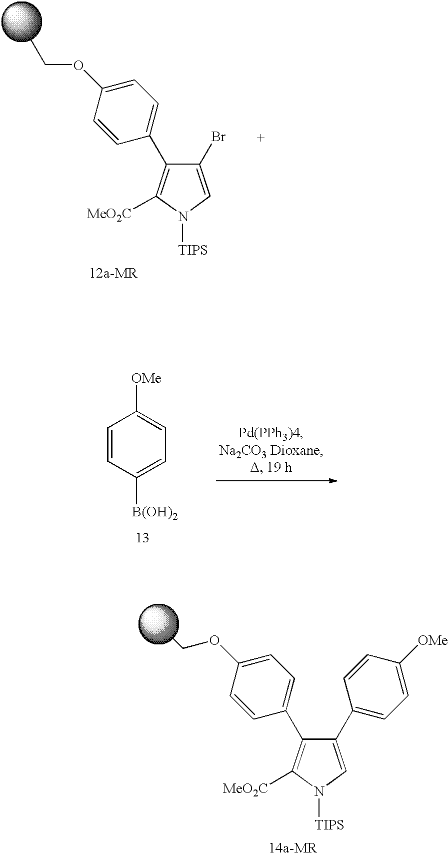

The resin 12a-MR (150 mg, 0.07mmol, 0.46 mmol/g) was swelled with dioxane (5 mL) for 15 min. After this time aq. 2 M Na₂CO₃ (172 μL, 10 equiv.), 4-methoxyboronic acid 13 (41.9 mg, 0.27 mmol, 10 equiv.) and Pd(PPh₃)₄ (8 mg, 0.007 mmol, 0.2 equiv.) were added and the reaction mixture was refluxed for 19 h. After this time the resin 14a-MR was washed with dioxane, DCM, MeOH, Et₂O (3×4 mL, each), and was dried at vacuum.

¹³C NMR (75 MHz, Gel Phase, CDCl₃) δ 13.6 (CH(CH₃)₂), 18.6 (CH(CH₃)₂), 53.4 (CO₂CH₃), 67.0 (OCH₃).

### Example 13

### Methyl 3-(4-hydroxyphenyl)-4-(4-methoxyphenyl)-1H-pyrrole-2-carboxylate 14b (cleavage with AlCl₃)

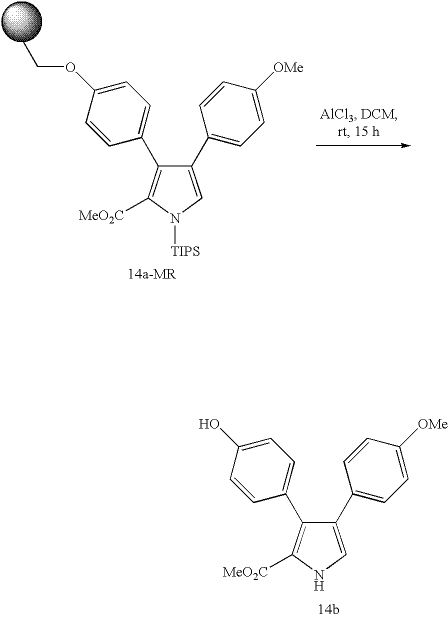

The resin 14a-MR (600 mg, 0.46 mmol/g) was swelled in DCM (30 mL) under N₂. AlCl₃ (560 mg, 4.16 mmol) was added and the reaction was stirred in the vibromatic 3ht at room temperature. After this time the resin was washed with DCM. The organic solution was washed with 10% HCl, dried over Na₂SO₄ and evaporated. The crude was analysed by HPLC-MS (C18-ES⁺ using H₂O (0.04% formic acid): MeCN (0.06% formic acid) gradient 30-70 % MeCN in 15 min): the pyrrole 14b has a tr 5.67 min. MS: 324.1. Purification by HPLC (gradient 2065% AcCN in 40 min) gave 14b (1 mg, 2%) as a pale-yellow solid.

¹H NMR (CDCl₃, 500 MHz) δ 3.73 (s, 3H, CO₂CH₃), 3.76 (s, 3H, OCH₃), 6.76 (bt, J 8.7, 4H, H3′, H5′, H3″, H5″), 7.00-7.06 (m, 3H, H2′, H6′, H5), 7.14 (d, J 8.5, 2H, H2″, H6″), 9.12 (bs, NH/OH).

¹³C NMR (CDCl₃, 125 MHz) δ 51.4 (s, CO₂CH₃), 55.2 (s, OCH₃), 113.8 (d, C3″, C5′), 114.8 (d, C3′, C5′), 129.6 (d, C2′, C6′), 132.2 (d, C2″, C6″).

MS (EI) m/z 324 (M+1, 12), 323 (M⁺, 9), 292 (18).

HRMS (EI) m/z calculated for C₁₉H₁₇NO₄: 323.1157; found: 323.1157.

### Example 14

### 4-(2-methoxycarbonyl-4-(4-methoxyphenyl)-1H-pyrrol-3-yl)-phenoxy-resin 15-MR

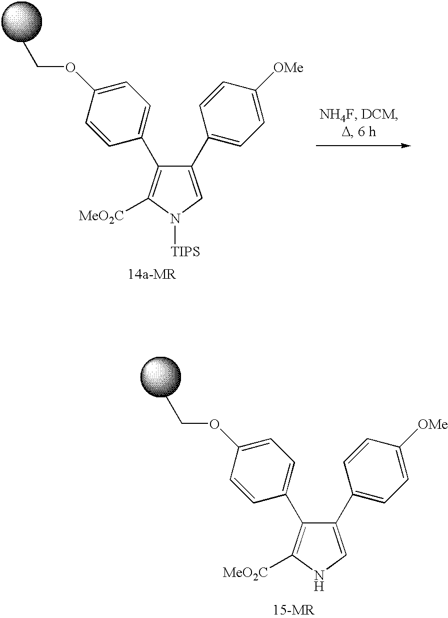

The resin 14a-MR (2 g, 0.8 mmol, 0.44 mmol/g) was swelled in DCM (30 mL) for 10 min and NH₄F (227 mg, 6.14 mmol, 7 equiv.) and MeOH (30 mL) were added and the mixture was shaken in the vibromatic and refluxed for 6 h. After this time the resin was washed with DCM and MeOH (5×4 mL, each), and was dried.

¹³C NMR (75 MHz, Gel Phase, CDCl₃) δ 53.4 (CO₂CH₃), 69.8 (OCH₃).

IR (KBr, cm⁻¹) 3430, 1690.

### Example 15

### Methyl 3-(4-hydroxyphenyl)-4(4-methoxyphenyl)-1-(2-(4-methoxyphenyl)-2-ethanone-yl)-1H-pyrrole-2-carboxylate: DimethoxyLamellarin O

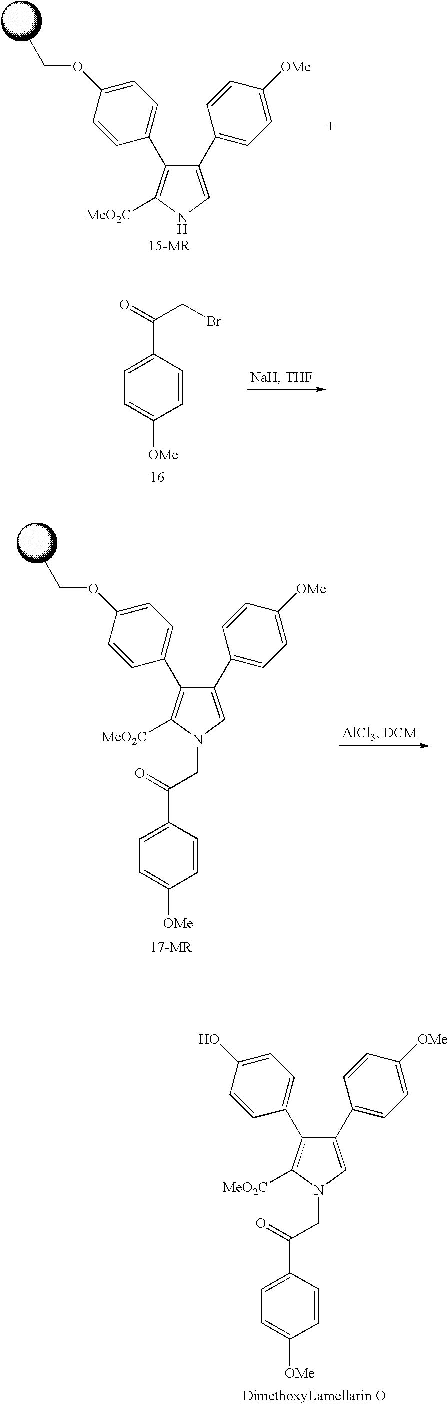

2-bromo-1-(4-methoxyphenyl)ethanone 16 (548 mg, 2.39 mmol, 19 equiv.) and NaH (75 mg, 1.89 mmol, 15 equiv.) were added to the swelled resin 15-MR (350 mg, 0.126 mmol, 0.36 mmol/g) in dry THF (20 mL) under N₂,. The reaction mixture was shacked in the vibromatic at 128° C. for 23 h. After this time the resin was washed with dioxane, DCM, MeOH and Et₂O (3×3mL, each). After this washing process the resin was dried at vacuum.

Following the cleavage conditions described in the example 6, from 17-MR (332 mg, 0.119 mmol, 0.36 mmol/g) the DimethoxyLamellarin O (R₁═R₂═R₄═R₅═R₆═R₇═R₉═R₁₀═R₁₂═R₁₃═R₁₅═R₁₆═H, R₃═OH, R₈═R₁₄═OMe, R₁₁═ ═O) was obtained. The crude product was analysed by HPLC-MS (C18-ES⁺ with H₂O (0.04% formic acid): MeCN (0.06% formic acid) gradient 35-50% MeCN in 15 min): DimethozyLamellin O has a tr 14.94 min, MS: 472, (M+1).

### Example 16

### 5-(2-Formyloxy-4-isopropoxy-5-methoxy-phenylethynyl)-2-methoxy-phenoxy-resin 6a-MR

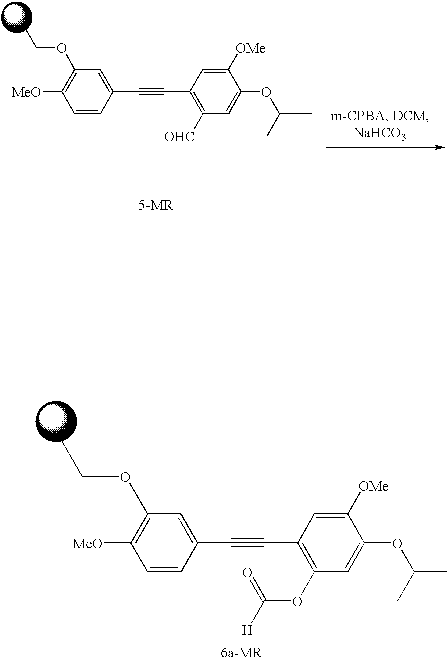

The resin 5-MR (480 mg, 0.49 mmol/g) was swelled in DCM for 30 minutes and NaHCO₃ (237 mg, 2.80 mmol, 12 equiv.) was added in one portion. The mixture was cooled in an ice bath and m-CPBA (483 mg, 1.4 mmol, 6 equiv.) was added in 3 portions in 3 h. After that time the reaction mixture was shaken overnight at room temperature. The solid phase was filtered and washed with DCM, DMF, DMF:H₂O (1:1), H₂O and MeOH (5×15 mL, each). The filtered resin was dried under reduce pressure.

IR (KBr, cm⁻) v 1730 (C═O).

### Example 17

### 5-(2-Formyloxy-4-isopropoxy-5-methoxyphenylethynyl)-2-methoxy-phenoxy-resin 6a-WR

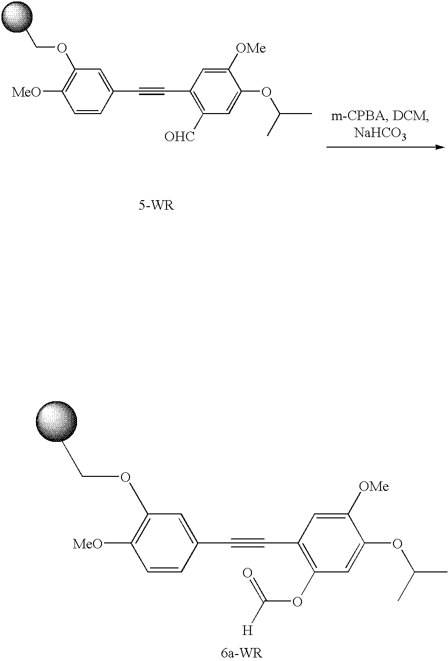

The resin 5-WR (1.14 g, 0.67 mmol/g) was swelled in DCM for 30 minutes and NaHCO₃ (0.91 g, 10.08 mmol, 12 equiv.) was added in one portion. The mixture was cooled in an ice bath and m-CPBA (1.86 g, 5.4 mmol, 6 equiv.) was added in 3 portions in 3 h. After that time the reaction mixture was shaken overnight at room temperature. The solid phase was filtered and washed with DCM, DMF, DMF:H₂O (1:1), H₂O, and MeOH (5×20 mL, each). The filtered resin was dried under reduce pressure.

IR (KBr, cm⁻) v 1730 (C═O).

### Example 18

### 5-(2-Hydroxy-4-isopropoxy-5-methoxyphenylethynyl)-2-methoxyphenoxy-resin 6b-MR

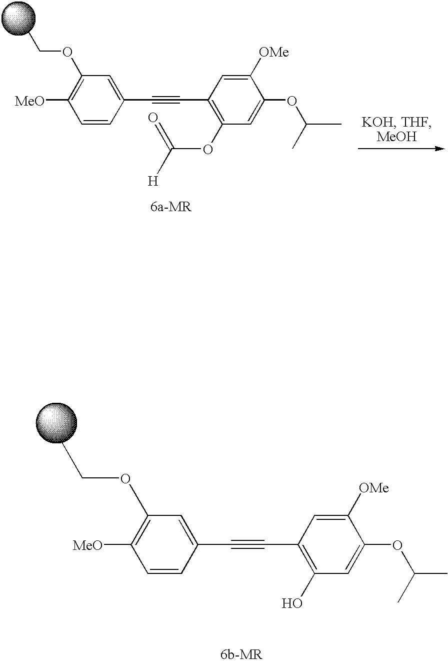

The resin 6a-MR (480 mg, 0.49 mmol/g) was swelled in THF for 15 min and 2 M KOH in MeOH/THF (1:1, 6 mL) was added. The mixture was shaken for 5 h at room temperature. The resin was filtered and washed with THF, THF: H₂O (1:1), H₂O, MeOH, DCM, DMF, THF, Et₂O (5×15 mL, each) and finally was dried under reduced pressure.

IR (KBr, cm⁻¹) v 3417 (OH).

¹³C NMR (Gel Phase, 75 MHz, CDCl₃) δ 71.1 (OCH(CH₃)₂), 56.5 (OCH₃), 21.9 (CH(CH₃)₂).

### Example 19

### 5-(2-Hydroxy-4-isopropoxy-5-methoxyphenylethynyl)-2-methoxyphenoxy-resin 6-WR

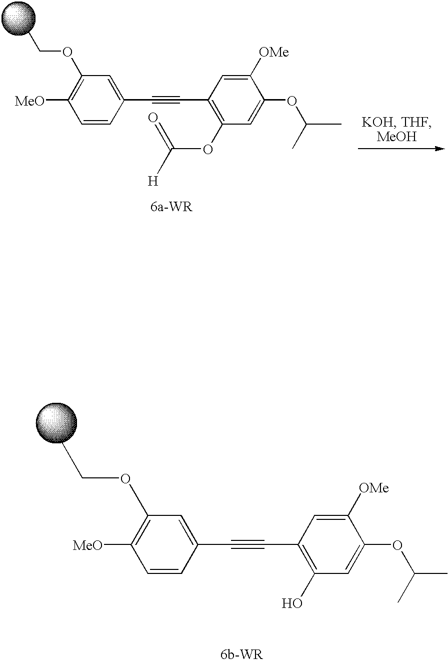

The resin 6a-WR (1.14 g, 0.67 mmol/g) was swelled in THF for 15 min and 2 M KOH in MeOH:THF (1:1, 12 mL) was added. The mixture was shaken for 5 h at room temperature. The resin was filtered and washed with THF, THF:H₂O (1:1), H₂O, MeOH, DCM, DMF, THF, Et₂O (5×20 mL, each) and finally was dried under reduced pressure.

IR (KBr, cm⁻¹) v 3417 (OH).

¹³C NMR (Gel Phase, 75 MHz, CDCl₃) δ 71.1 (OCH(CH₃)₂), 56.5 (OCH₃), 21.9 (CH(CH₃)₂).

### Example 20

### 5-(2-Iodoacetoxy)-4-isopropoxy-5-methoxyphenylethynyl)-2-methoxy-phenoxy-resin 7-MR

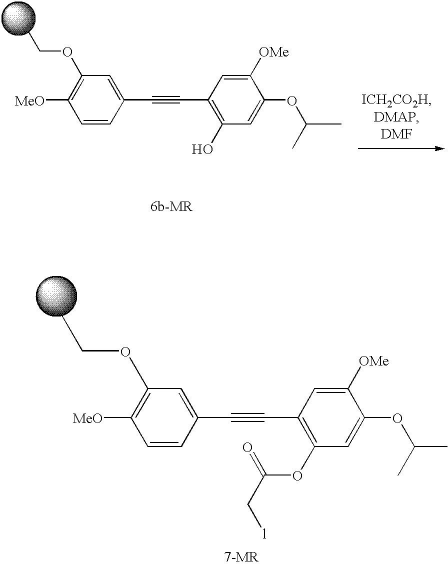

The resin 6b-BE (240 mg, 0.49 mmol/g) was swelled in 5 mL of dry DMF for 30 minutes. After this time 2-iodoacetic acid (223 mg, 1.2 mmol, 10 equiv.), DMAP (22 mg, 0.18 mmol, 15%) and DIPAD (151 mg, 1.2 mmol, 10 equiv.) were added. The resulting mixture was shaken under Ar for 12 h. The reaction was filtered and the collected solid was washed with DMF, THF, DCM, Et₂O (5×10 mL, each) and finally was dried under reduce pressure. The reaction was repeated under the same conditions to achieve better yields;

IR (KBr, cm⁻¹) v 2200 (C≡C), 1735 (C═O).

¹³C MAS-NMR(125 MHz, CDCl₃) δ 148.1, 148.0, 145.9, 145.8, 145.2, 144.7, 114.9, 108.5, 82.4 (Ar—C≡C—Ar), 71.6 (OCH(CH₃)₂), 56.1 (OCH₃), 55.8 (OCH₃), 21.8 (OCH(CH₃)₂), −5.46 (CH₂).

### Example 21

### 5-(2-(2-Iodo-acetoxy)-4-isopropoxy-5-methoxyphenylethynyl)-2-methoxy-phenoxy-resin 7-WR

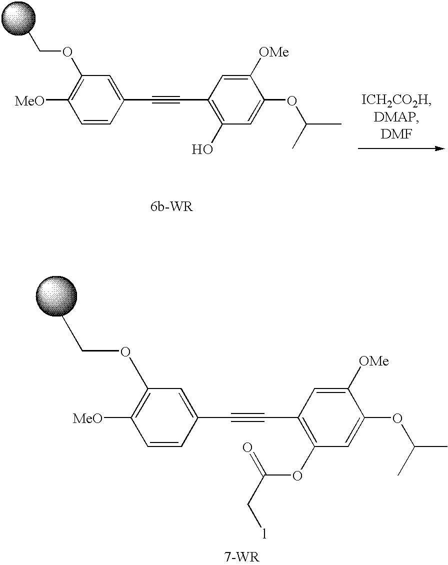

The resin 6b-WR (630 mg, 0.67 mmol/g) was swelled in 8 mL of dry DMF for 30 minutes. After this time 2-iodoacetic acid (471 mg, 2.53 mmol, 6equiv.), DMAP (46 mg, 0.38 mmol, 15% respect iodoacetic acid) and DIP (319 mg, 2.53 mmol, 6 equiv.) were added. The resulting mixture was shaken under Ar for 12 h. The reaction was filtered and the collected solid was washed with DMF, THF, DCM, Et₂O (5×15 mL, each) and finally was dried under reduce pressure. The reaction was repeated under the same conditions to achieve better yields.

IR (KBr, cm⁻¹) v 2200 (C≡C), 1735 (C═O).

¹³C MAS-NMR(125 MHz, CDCl₃) δ 148.1, 148.0, 145.9, 145.8, 145.2, 144.7, 114.9, 108.5, 82.4 (Ar—C≡C—Ar), 71.6 (OCH(CH₃)₂), 56.1 (OCH₃), 55.8 (OCH₃), 21.8 (OCH(CH₃)₂), -5.46 (CH₂).

### Example 22

### 5-(8,9-Dihydro-3-isopropoxy-2,11,12-trimethoxy-6H-[1]benzopyrano[4′,3′:4,5]pyrrolo[2,1-α]isoquinolin-6-one-14-yl)-2-methoxy-phenoxy-resin 9-MR

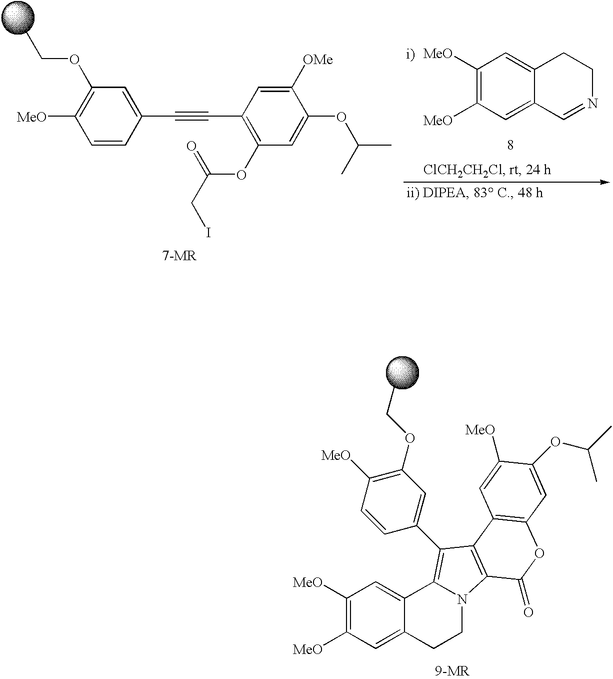

The iodoesther Merrifield resin 7-MR (120 mg, 0.45 mmol/g) was swelled in dry 1,2-dichloroethane (5 mL) and 3,4-dihydro-6,7-dimethoxyisoquinoline 8 (69 mg, 0.36 mmol, 6 equiv.) was added in two portions. The reaction mixture was shaken for 24 h at room temperature. After this time DIPEA (107 mg, 7 equiv.) was added and the mixture was heated at 83° C. for 48 h. The reaction mixture was filtered and washed with DCM, DMF, THF, Et₂O (5×5 mL, each). The resin was dried under reduce pressure.

IR (KBr, cm⁻¹): v 1749 cm⁻(C═O).

### Example 23

### 5-(3-Isopropoxy-8,9-dihydro-2,11,12-trimethoxy-6H-[1]benzopyrano[4′,3′:4,5]pyrrolo[2,1-α]isoquinolin-6-one-1 4-yl)-2-methoxy-phenoxy-resin 9-WR

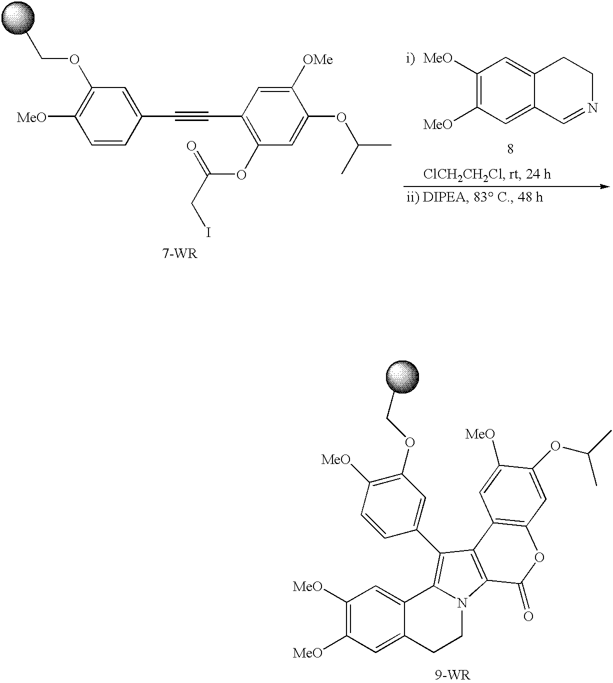

The Wang resin 7-WR (645 mg, 0.60 mmol/g) was swelled in dry 1,2-dichloroethane (10 mL) and 3,4-dihydro-6,7-dimethoxyisoquinoline 8 (520 mg, 2.71 mmol, 7 equiv.) was added in two portions. The reaction mixture was shaken for 24 h at room temperature. After this time DIPEA (470 mg, 7 equiv.) was added and the mixture was heated at 83° C. for 48 h. The reaction mixture was filtered and washed with DCM, DMF, THF, Et₂O (5×15 mL, each). The resin was dried under reduce pressure.

IR (KBr, cm⁻¹): v 1749 cm⁻¹ (C═O).

### Example 24

### 3-Acetoxy-8,9-dihydro-2,11,12-trimethoxy-14-(3′-acetoxy-4′-methoxy-phenyl)-6H-[1]benzopyrano[4′,3′:4,5]pyrrolo[2,1-a]isoquinolin-6-one: 3′,3-Di-O-acetyllamellarin U

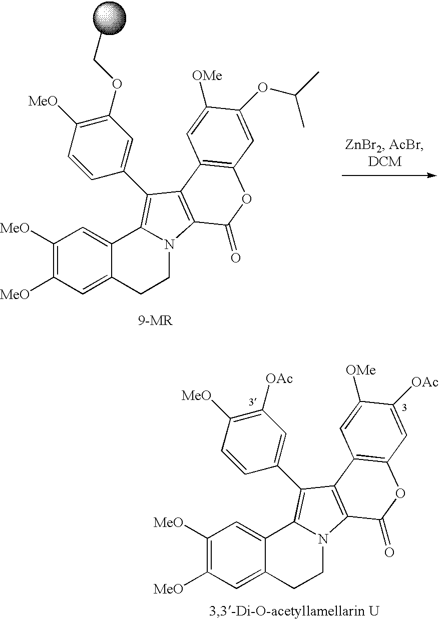

The general procedure described in example 5 from 9-MR (180 mg, 0.48 mmol/g theoretical loading), dry ZnBr₂ (225 mg, 4 equiv.) and acetylbromide (111 mg, 10 equiv.) was followed. The crude was analyzed by HPLC-MS (C18-APCI⁺ using H₂O (5 mM AcNH₄): MeCN gradient 30-100% MeCN in 15 min): tr 9.73 min, calcd 599.58; found 600.6 (M+H)⁺. MSMS (Q-TOF) calcd 599.18; found 600.20 (M+H)⁺, 557, 22 ((M+H)⁺—C₂H₂O), 515.29 ((M+H)⁺-2×C₂H₂O). The crude product was purified by HPLC and 3, 3′-Di-O-acetylLamellarin U (4 mg, 8.5%) was obtained.

¹H NMR (600 MHz, CDCl₃) δ 7.30 (dd, J=8.2 and 2.0 Hz, 1H), 7.22 (d, J=2.0 Hz, 1H, H-16), 7.12 (d, J=8.2 Hz, 1H), 7.06 (s, 1H), 6.74 (s, 1H), 6.72 (s, 1H), 6.62 (s, 1H), 4.95 (m, 1H), 4.64 (m, 1H), 3.87 (s, 6H), 3.44 (s, 3H), 3.40 (s, 3H), 3.15 (m, 1H), 3.05 (s, 1H), 2.29 (s, 3H), 2.27 (s, 3H).

¹³C NMR (150 MHz, CDCl₃) δ 168.8 (s), 168.3 (s), 151.2 (s), 149.0 (s), 147.5 (s), 147.4 (s), 144.8 (s), 140.7 (s), 138.7 (s), 136.1 (s), 129.5 (d), 127.7 (s), 127.4 (s), 126.4 (s), 125.0 (d), 119.8 (s), 116.1 (s), 114.1 (s), 112.5 (d), 111.8 (d), 110.9 (d), 108.5 (d), 105.4 (d), 56.0 (c), 56.0 (c), 55.6 (c), 55.2 (c), 42.5 (t), 28.7 (t), 20.0 (c), 20.0 (q).

(+)-HRMS m/z 599.1789 (callcd for C₃₃H₂₉NO₁₀ (M)⁺ 599.1791, Δ+0.4 ppm).

### Example 25

### 7,8-Dihydro-3-hydroxy-2,11,12-trimethoxy-14-(3′-hydroxy-4′-methoxy-phenyl)-6H-[1]benzopyrano[4′,3′:4,5]pyrrolo[2,1-α]isoquinolin-6-one: Lamellarin U, 8,9-dihydro-3,11-dihydroxy-2,12-dimethoxy-14-(3′-hydroxy-4′-methoxy-phenyl)-6H-[1]benzopyrano[4′,3′:4,5]pyrrolo[2,1-α]isoquinolin-6-one: Lamellarin L, and 8,9-dihydro-3,11-dihydroxy-2,12-dimethoxy-14-(3′-hidroxy-4′-methoxy-phenyl)-6H-[1]benzopyrano[4′,3′:4,5]pyrrolo[2,1-α]isoquinolin-6-one: 11-Demethyllamellarin U

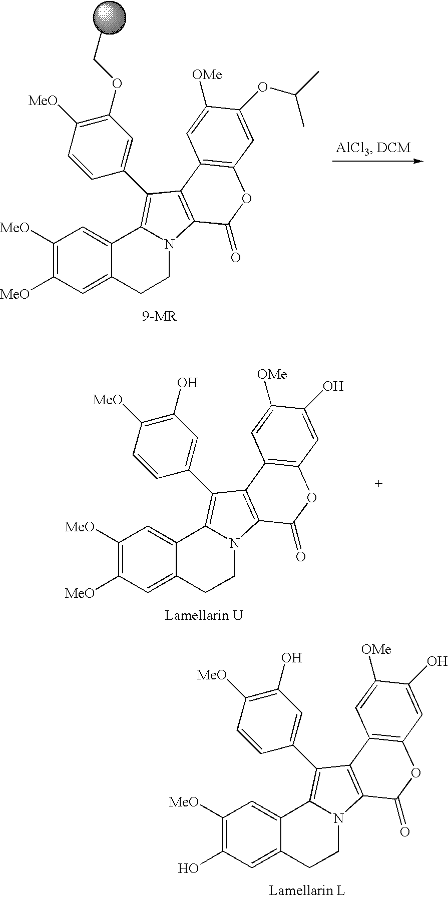

The resin 9-MR (220 mg, 0.44 mmol/g loading) was swelled in dry DCM (3 mL) for 30 min and AlCl₃ (220 mg, 1.65 mmol, 15 equiv.) was added. The reaction mixture was stirred in a vibromatic shaker at 25° C. for 6 h. It was then filtered and washed with DCM, EtOAc and MeOH (5×10 mL, each), the organic solvent was evaporated. The residue was taken with a sat. aq. solution of NH₄Cl and extracted with ethylacetate (5×20 mL), then washed with brine (1×30 mL). The organic fraction was dried and evaporated.

The HPLC/MS (C18-APCI⁺ using H₂O (5 mM AcNH₄): MeCN gradient 30-100% MeCN in 15 min) shown three lamellarin derivatives: lamellarin U, tr 8.0 min, calcd 515.16; found 516.19 (M+H)⁺; 11-DemethylLamellarin U., tr 6.9 min, calcd 501.14, found, 502.15 (M+H)⁺, and lamellarin L, tr 6.6 min, calcd 501.14, found 502.15 (M+H)⁺. The crude product was purified by HPLC: lamellarin U (4.5 mg, 9.2%), 11-DemethylLamellarin U (1 mg, 2.0%), and lamellarin L (1.5 mg, 3.1%) were obtained.

### Lamellarin U

¹H NMR (600 MHz, CDCl₃) δ 7.11 (d, J=1.9 Hz, 1H), 7.02 (d, J=8.2 Hz, 1H), 6.99 (dd, J=8.2 and 1.9 Hz, 1H), 6.94 (s, 1H), 6.73 (s, 1H), 6.70 (s, 1H), 6.69 (s, 1H), 5.69 (s, 1H), 5.66 (s, 1H), 4.80 (m, 1H), 4.72 (m,1H), 3.95 (s, 3H), 3.87 (s, 3H), 3.51 (s, 3H), 3.37 (s, 3H), 3.08 (dd, J=6.6 and 5.7 Hz, 2H).

¹³C NMR (150 MHz, CDCl₃) δ 151.4 (s), 149.3 (s), 147.9 (s), 146.8 (s), 145.8 (s), 143.6 (s), 136.3 (s), 129.2 (s), 128.6 (s), 127.1 (s), 123.4 (d), 120.5 (s), 117.4 (d), 114.9 (d), 111.1 (d), 110.9 (d), 110.7 (d),108.8 (d), 104.1 (d), 103.3 (d), 56.2 (q), 55.8 (q), 55.5 (q), 55.0 (q), 42.8 (t), 28,6 (t)

(+)-HRMS m/z 516.1683 (calcd for C₂₉H2₂₆NO₈ (M+H)⁺ 516.1166, Δ−4.8 ppm).

### Lamellarin L

¹H NMR (600 MHz, CDCl₃) δ 7.11 (d, J=1.9 Hz, 1H), 7.02 (d, J=8.3 Hz, 1H), 6.98 (dd, J=8.3 and 1.9 Hz, 1H), 6.93 (s, 1H), 6.79 (s, 1H), 6.68 (s, 1H), 6.67 (s, 1H), 5.70 (bs, 2H), 5.60 (bs, 1H), 4.78 (m, 1H), 4.70 (m, 1H), 3.96 (s, 3H), 3.50 (s, 3H), 3.41 (s, 3H), 3.01 (m, 2H).

¹³C NMR (150 MHz, CDCl₃) δ 146.4 (s), 146.3 (s), 146.2 (s), 145.7 (s), 145.1 (s), 143.2 (s), 135.4 (s), 128.3 (s), 128.2 (s), 127.4 (s), 122.9 (d), 119.8 (s), 117.3 (d), 114.0 (d), 111.9 (d), 108.4 (d), 104.1 (d), 103.5 (d), 56.2 (q), 55.5 (q), 55.1 (q), 42.2 (t), 28.2 (t).

### 11-Demethyllamellarin U

¹H NMR (600 MHz, CDCl₃) δ 7.04 (d, J=2.0 Hz, 1H), 7.00 (d, J=8.0 Hz, 1H), 6.94 (dd, J=8.0 and 2.0 Hz, 1H), 6.92 (s, 1H), 6.74 (s, 1H), 6.73 (s, 1H), 6.57 (s, 1H), 5.68 (s, 1H), 5.65 (s, 1H), 5.36 (s, 1H), 4.80-4.72 (m, 2H), 3.97 (s, 3H), 3.89 (s, 3H), 3.49 (s, 3H), 3.07 (m, 2H).

¹³C NMR (150 MHz, CDCl₃) δ 146.3, 146.2, 146.2, 145.2, 144.9, 143.8, 142.9, 135.2, 128.1, 126.1, 122.8, 120.6, 117.1, 115.7, 113.1, 112.1, 111.2, 110.3, 104.1, 103.2, 55.9, 55.7, 55.3, 42.3, 28.8.

(+)-HRMS m/z 502.1508 (calcd for C₂₈H₂₄NO₈ (M+H)⁺ 502.1502, Δ−1.1 ppm).

### Example 26

### 7,8-Dihydro-3-isopropoxy-2,10,11-trimethoxy-13-(3′-hydroxy-4′-methoxy-phenyl)-6H-[1]benzopyrano[4′,3′:4,5]pyrrolo[2,1-α]isoquinolin-6-one: 3-O-Isopropyllamellarin U and 7,8-Dihydro-3-isopropoxy-2,10,11-trimethoxy-13-(2-chloro-4′-methoxy-5′-hydroxy-phenyl)-6H-[1]benzopyrano[4′,3′:4,5]pyrrolo[2,1-α]isoquinolin-6-one: 2′-Chloro-3-O-isopropyllamellarin U

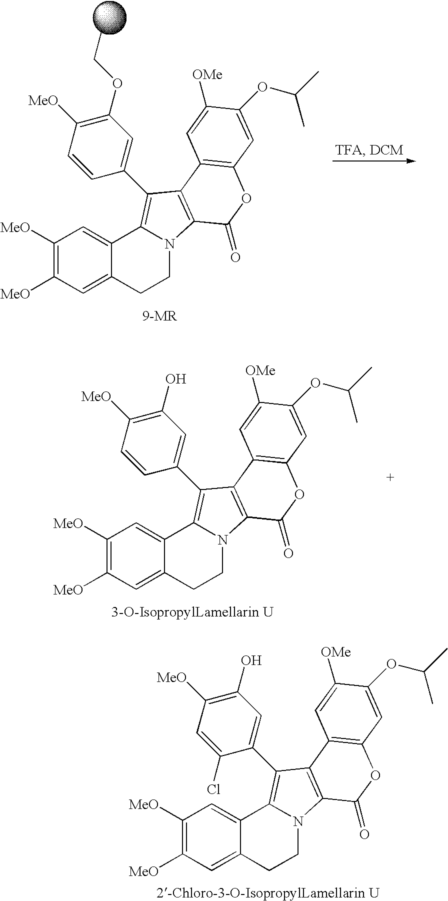

Following the general procedure described in Example 7 from 9-WR (300 mg) a reaction crude was obtained. The HPLC-MS (C18-APCI⁺ using H₂O (5 mM AcNH₄): MeCN gradient 30-100% MeCN in 15 min) shown two different lamellarins derivatives: 2′-Chloro-3-O-isopropylLamellarin U tr 11.07 min, calcd 591.17, found, 592.23 (³⁵Cl M+H)⁺, 594.23 (³⁷Cl M+H)⁺, 3-O-isopropylLamellarin U tr 11.3 min, calcd 557.20; found 558.30 (M+H)⁺. The crude product was purified by HPLC: 2′-Chloro-3-O-isopropylLamellarin U (2 mg, 2%), and 3-O-isopropylLamellarin U (8.5 mg, 9%) were obtained.

### 3-O-Isopropyllamellarin U:ç

¹H NMR (600 MHz, CDCl₃) δ 7.11 (d, J=2.0 Hz, 1H), 7.14 (d, J=8.3 Hz, 1H), 6.99 (dd, J=8.3 and 2.0 Hz, 1H), 6.90 (s, 1H), 6.73 (s, 1H), 6.72 (s, 1H), 6.71 (s, 1H), 5.68 (bs, 1H), 4.83 (m, 1H), 4.74 (m, 1H), 4.52 (sept, 1H), 3.94 (s, 3H), 3.87 (s, 3H), 3.45 (s, 3H), 3.37 (s, 3H), 3.09 (m, 2H), 1.37 (s, 3H), 1.36 (s, 3H).

¹³C NMR (150 MHz, CDCl₃) δ 148.8 (C), 147.4 (s), 147.1 (s), 146.4 (s), 146.3 (s), 146.1 (s), 135.8 (s), 128.6 (s), 128.1 (s), 126.5 (s), 122.9 (d), 120.1 (s), 117.3 (d), 114.7 (C) 111.2 (d),110.9 (d), 110.3 (s), 108.8 (d), 105.1 (d), 103.5 (d), 71.4 (d), 56.2 (q), 55.8 (q), 55.4 (q), 55.0 (q), 42.3 (t), 28.6 (t), 21.7 (q), 21.7 (q).

(+)-HRMS m/z 557.2067 (calcd for C₃₂H₃₁NO₈ (M)⁺ 557.2050, Δ−3.0 ppm).

The presence of the unexpected 2′-Chloro-3-O-isopropylLamellarin U was confirmed:

¹H NMR (600 MHz, CDCl₃) δ 7.11 (s, 1H), 7.07 (s, 1H) 6.90 (s, 1H), 6.75 (s, 1H), 6.62 (s, 1H), 6.56 (s, 1H), 5.61 (bs, 1H), 4.93 (m, 1H), 4.66 (m, 1H), 4.51 (hept, 1H), 3.96 (s, 3H), 3.87 (s, 3H), 3.48 (s, 3H), 3.42 (s, 3H), 3.09 (m, 2H) 1.38 (d, 6H).

¹³C NMR (150 MHz, CDCl₃) δ 148.9, 148.4, 147.1, 147.4, 146.9, 146.5, 145.8, 135.9, 128.3, 126.6, 126.2, 119.9, 118.6, 112.0, 110.9, 109.9, 108.2, 104.7, 103.3, 71.3, 56.4, 55.7, 55.4, 55.0, 45.4, 28.6, 21.6.

(+)-HRMS m/z 592.1751 (calcd for C₃₂H₃₁NO₈C (M+H)⁺ 592.1738, Δ−2.2 ppm.

### Example 27

### 5-Iodo-4-methyl-2-methoxyphenoxy-resin 18-MR

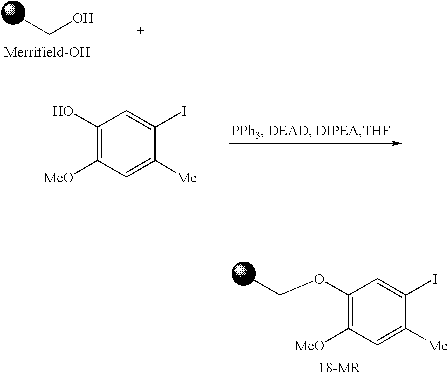

Merrifield-OH resin (4.0 g of 100-200 mesh material, 0.98 mmol/g loading) was washed with DCM (1×40 mL) and THF (1×40 mL) and dried. To the swelled resin with THF (50 mL), 5-iodo-2-methoxy-4-methyl-phenol (4.12 g, 15.68 mmol, 4 equiv.), PPh₃ (4.10 g, 15.68 mmol, 4 equiv.), TEA (8.1 mL, 47.04 mmol, 12 equiv.) were added and the resulting mixture was shaken at 0° C. for 10 min. DEAD (2.5 mL, 15.68 mmol, 4 equiv.) was added dropwise and the resulting mixture was shaken at room temperature over nigth. The solvent was then removed. The resin was washed with DMF, DCM, MeOH, and Et₂O (5×50 mL, each). The same treatment was repeated. The resin was dried under reduced pressure. IR (KBr) Disappearance of the stretching band at 3576 and 3440 cm⁻¹.

### Example 28

### 2-Iodo-6-methylpyridin-3-yloxy-resin 19-HE

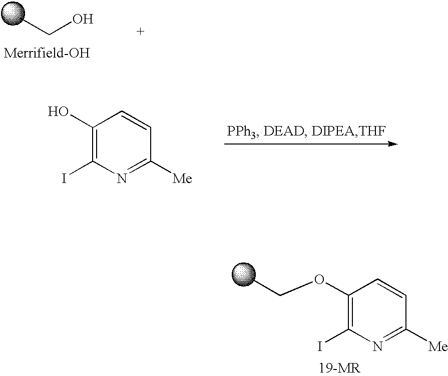

Merrifield-OH resin (4.0 g of 100-200 mesh material, 0.98 mmol/g loading) was washed with DCM (1×40 mL) and THF (1×40 mL) and dried. To the swelled resin with THF (50 mL), 2-iodo-6-methylpyridin-3-ol (2.76 g, 11.76 mmol, 3 equiv.), PPh₃ (3.09 g, 11.76 mmol, 3 equiv.), TEA (6.0 mL, 35.28 mmol, 9 equiv.) were added and the resulting mixture was shaken at 0° C. for 10 min. DEAD (2.1 mL, 11.76 mmol, 3 equiv.) was added dropwise and the resulting mixture was shaken at room temperature over night. The solvent was then removed. The resin was washed with DMF, DCM, MeOH, and Et₂O (5×50 mL each). The same reaction process was repeated. The resin was dried under reduced pressure. IR (KBr) Disappearance of the stretching band at 3576 and 3440 cm⁻¹.

### Example 29

### 4-Iodo-2-methylphenoxy-resin 20-MR

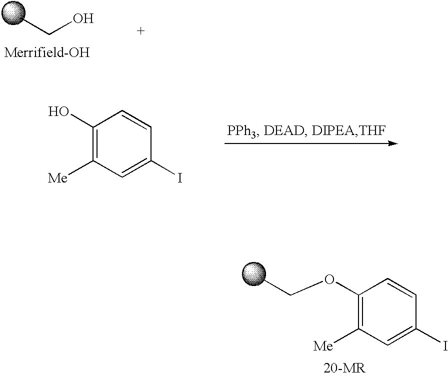

Merrifield-OH resin (4.0 g of 100-200 mesh material, 0.98 mmol/g loading) was washed with DCM (1×40 mL) and THF (1×40 mL) and dried. To the swelled resin with THF (50 mL), 4-iodo-2-methylphenol (3.67 g, 15.68 mmol, 4 equiv.), PPh₃ (4.10 g, 15.68 mmol, 4 equiv.), TEA (8.1 mL) were added and the resulting mixture was shaken at 0° C. for 10 min. DEAD (2.5 mL, 15.68 mmol, 4 equiv.) was added dropwise and the resulting mixture was shaken at room temperature over nigth. The solvent was then removed. The resin was washed with DMF, DCM, MeOH, and Et₂O (5×50 mL each). The resin was dried under reduced pressure. IR (KBr) Disappearance of the stretching band at 3576 and 3440 cm⁻¹.

### Example 30

### 5-(5-Fluoro-2-formylphenylethynyl)-2-methoxyphenoxy-resin 21-MR

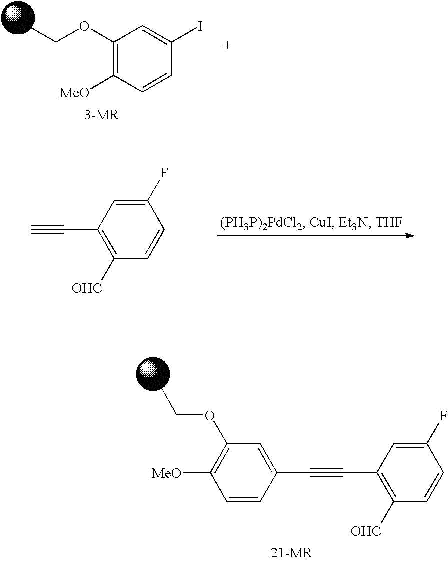

A solution of 2-ethynyl-4-fluorobenzaldehyde (0.93 g, 6.3 mmol, 4 equiv.) in THF and CuI (180 mg, 0.95 mmol, 0.6 equiv.) was added to Merrifield resin 3-MR (2 g, 0.78 mmol/g loading) swelled with a mixture of THF:TEA (3:1) for 30 min. The resulting suspension was bubbled with N₂ for 10 min and (Ph₃P)₂PdCl₂ (330 mg, 0.47 mmol, 0.3 equiv.) was added and the reaction mixture was shaken at room temperature for 20 h. The resin was filtered and washed with THF, DMF, 0.02 M sodium dihexyldithiocarbamate in DMF, DMF, THF, DCM, MeOH (5×15 mL, each) and dried under reduce pressure. The same reaction process was repeated.

IR (KBr, cm⁻¹) v 2200 (C≡C), 1687 (HC═O).

¹⁹F NMR (300 MHz, Gel Phase, CDCl₃) δ −110.8.

### Example 31

### 5-(2-Formyl-4-Isopropoxy-5-methoxyphenylethynyl)-2-methoxy-4-methyl-phenoxy-resin 22-MR

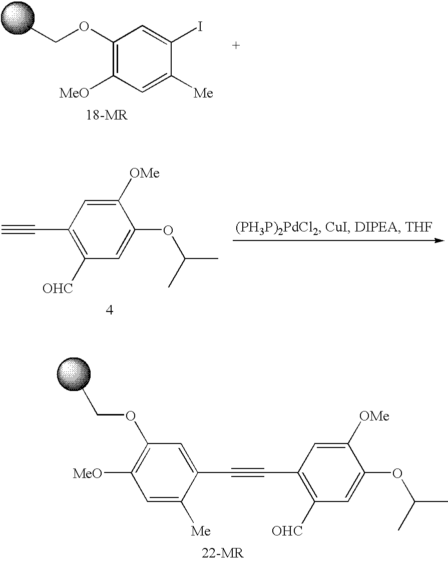

A solution of 2-ethynyl-5-isopropoxy-4-methoxybenzaldehyde 4 (1.36 g, 6.2 mmol, 4 equiv.) in THF and CuI (177 mg, 0.93 mmol, 0.6 equiv.) was added to Merrifield resin 18-MR (2.0 g, 0.78 mmol/g loading) swelled with a mixture of THF:TEA (3:1) for 30 min. The resulting suspension was bubbled with N₂ for 10 min and (Ph₃P)₂PdCl₂ (330 g, 0.47 mmol, 0.3 equiv.) was added and the reaction mixture was shaken at room temperature for 20 h. The resin was filtered and washed with THF, DMF, 0.02 M sodium dihexyldithiocarbamate in DMF, DMF, THF, DCM, MeOH (5×15 mL, each) and dried under reduce pressure. The same reaction process was repeated.

IR (KBr, cm⁻¹) v 2193 (C≡C), 1681 (HC═O).

### Example 32

### 5-(5-Fluoro-2-formyl-phenylethynyl)-2-methoxy-4-methyphenoxy-resin 23-MR

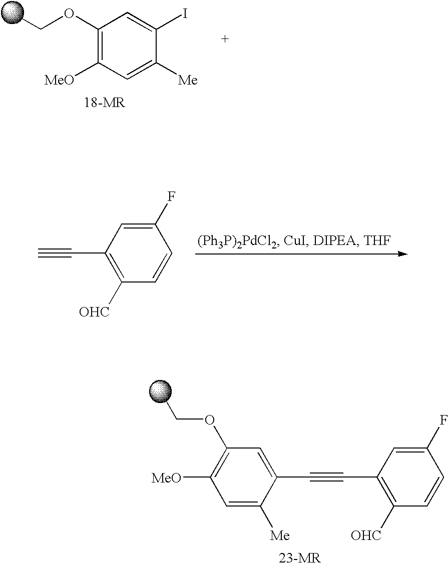

A solution of 2-ethynyl-4-fluorobenzaldehyde (0.92 g, 6.22 mmol, 4 equiv.) in THF and CuI (180 mg, 0.95 mmol, 0.6 equiv.) was added to Merrifield resin 18-MR (2 g, 0.78 mmol/g loading) swelled with a mixture of THF:TEA (3:1) for 30 min. The resulting suspension was bubbled with N₂ for 10 min and (Ph₃P)₂PdCl₂ (330 mg, 0.47 mmol, 0.3 equiv.) was added and the reaction mixture was shaken at room temperature for 20 h. The resin was filtered and washed with THF, DMF, 0.02 M sodium dihexyldithiocarbamate in DMF, DMF, THF, DCM, MeOH (5×15 mL, each) and dried under reduce pressure. The same reaction process was repeated.

IR (KBr, cm⁻¹) v 2200 (C≡C), 1684 (HC═O).

¹⁹F NMR (300 MHz, Gel Phase, CDCl₃) δ −111.0.

### Example 33

### 2-(2-Formyl-4-isopropoxy-5-methoxyphenylethynyl)-6-methylpyridin-3-yloxy-resin 24-MR

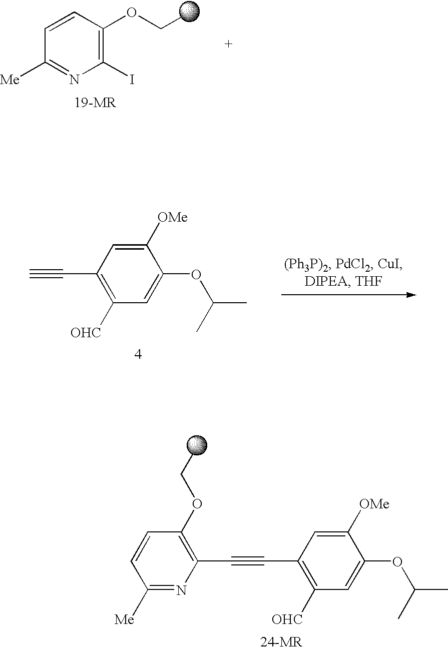

A solution of 2-ethynyl-5-isopropoxy-4-methoxybenzaldehyde 4 (1.39 g, 6.4 mmol, 4 equiv.) in THF and CuI (182 mg, 0.96 mmol, 0.6 equiv.) was added to Merrifield resin 19-MR (2.0 g, 0.79 mmol/g loading) swelled with a mixture of THF:TEA (3:1) for 30 min. The resulting suspension was bubbled with N₂ for 10 min and (Ph₃P)₂PdCl₂ (340 g, 0.48 mmol, 0.3 equiv.) was added and the reaction mixture was shaken at room temperature for 20 h. The resin was filtered and washed with THF, DMF, 0.02 M sodium dihexyldithiocarbamate in DMF, DMF, THF, DCM, MeOH (5×15 mL, each) and dried under reduce pressure. The same reaction process was repeated.

IR (KBr, cm⁻¹) v 2206 (C≡C), 1681 (HC═O).

### Example 34

### 2-(5-Fluoro-2-formyl-phenylethynyl)-6-methylpyridin-3-yloxy-resin 25-MR

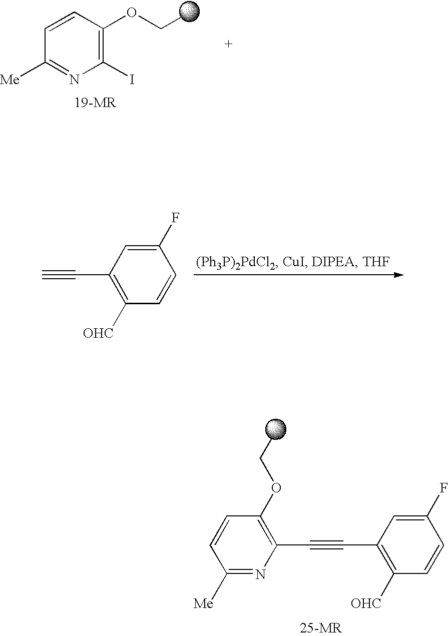

A solution of 2-ethynyl-4-fluorobenzaldehyde (0.94 g, 6.4 mmol, 4 equiv.) in THF and CuI (182 mg, 0.95 mmol, 0.6 equiv.) was added to Merrifield resin 19-MR (2 g, 0.79 mmol/g loading) swelled with a mixture of THF:TEA (3:1) for 30 min. The resulting suspension was bubbled with N₂ for 10 min and (Ph₃P)₂PdCl₂ (335 mg, 0.48 mmol, 0.3 equiv.) was added and the reaction mixture was shaken at room temperature for 20 h. The resin was filtered and washed with THF, DMF, 0.02 M sodium dihexyldithiocarbamate in DMF, DMF, THF, DCM, MeOH (5×15 mL, each) and dried under reduce pressure. The same reaction process was repeated.

IR (KBr, cm⁻¹) v 2206 (C≡C), 1687 (HC═O).

¹⁹F NMR (300 MHz, Gel Phase, CDCl₃) δ −109.5.

### Example 35

### 4-(2-Formyl-4-isopropoxy-5-methoxyphenylethynyl)-2-methylphenoxy-resin 26-MP

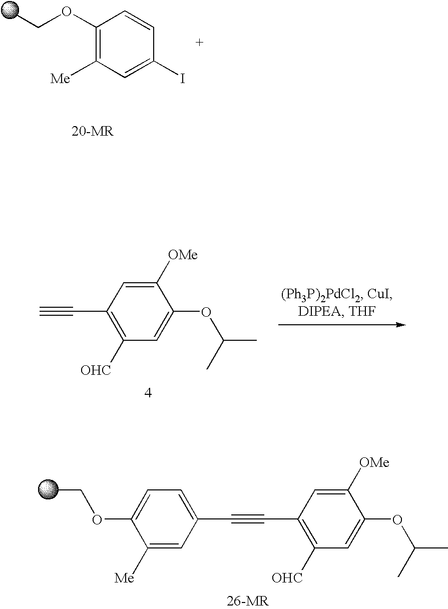

A solution of 2-ethynyl-5-isopropoxy-4-methoxybenzaldehyde 4 (1.39 g, 6.4 mmol, 4 equiv.) in THF and CuI (182 mg, 0.96 mmol, 0.6 equiv.) was added to Merrifield resin 20-MR (2.0 g, 0.79 mmol/g loading) swelled with a mixture of THF:TEA (3:1) for 30 min. The resulting suspension was bubbled with N₂ for 10 min and (Ph₃P)₂PdCl₂ (340 g, 0.48 mmol, 0.3 equiv.) was added and the reaction mixture was shaken at room temperature for 20 h. The resin was filtered and washed with THF, DMF, 0.02 M sodium dihexyldithiocarbamate in DMF, DMF, THF, DCM, MeOH (5×15 mL, each) and dried under reduce pressure. The same reaction process was repeated.

IR (KBr, cm⁻¹) v 2200 (C≡C), 1681 (HC═O).

### Example 36

### 4-(5-Fluoro-2-formylphenylethynyl)-2-methylphenoxy-resin 27-MR

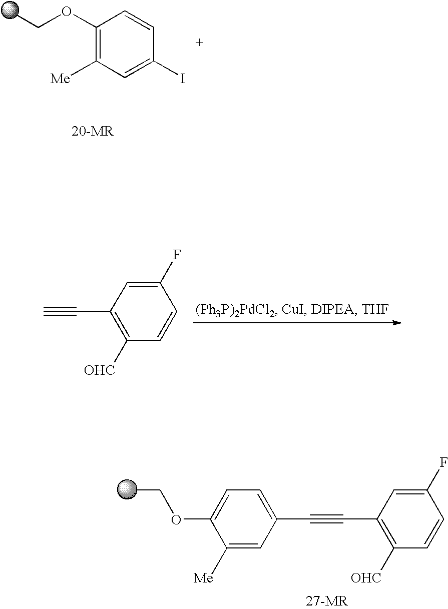

A solution of 2-ethynyl-4-fluorobenzaldehyde (0.94 g, 6.4 mmol, 4 equiv.) in THF and CuI (182 mg, 0.95 mmol, 0.6 equiv.) was added to Merrifield resin 20-MR (2 g, 0.79 mmol/g loading) swelled with a mixture of THF:TEA (3:1) for 30 min. The resulting suspension was bubbled with N₂ for 10 min and (Ph₃P)₂PdCl₂ (335 mg, 0.48 mmol, 0.3 equiv.) was added and the reaction mixture was shaken at room temperature for 20 h. The resin was filtered and washed with THF, DMF, 0.02 M sodium dihexyldithiocarbamate in DMF, DMF, THF, DCM, MeOH (5×15 mL, each) and dried under reduce pressure. The same reaction process was repeated.

IR (KBr, cm⁻¹) v 2200 (C≡C), 1687 (HC═O).

¹⁹F NMR (300 MHz, Gel Phase, CDCl₃) δ −111.1.

### Example 37

### 4-(4-Fluoro-2(-methoxymethoxy)phenylethynyl)-2-methoxyphenoxy-resin 28-MR

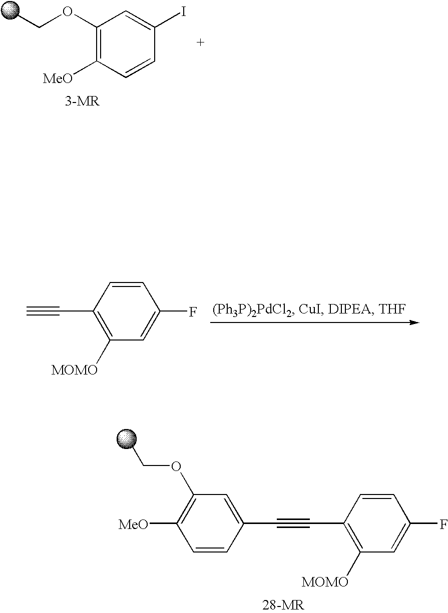

A solution of 1-ethynyl-4-fluoro-2-(methoxymethoxy)benzene (0.18 g, 1 mmol, 4.5 equiv.) in THF and CuI (25 mg, 0.13 mmol, 0.6 equiv.) was added to Merrifield resin 3-MR (0.30 g, 0.74 mmol/g loading) swelled with a mixture of THF:TEA (3:1) for 30 min. The resulting suspension was bubbled with N₂ for 10 min and (Ph₃P)₂PdCl₂ (46 mg, 0.06 mmol, 0.3 equiv.) was added and the reaction mixture was shaken at room temperature for 20 h. The resin was filtered and washed with THF, DMF, 0.02 M sodium dihexyldithiocarbamate in DMF, DMF, THF, DCM, MeOH (5×15 mL, each) and dried under reduce pressure. The same reaction process was repeated.

IR (KBr, cm⁻¹) v 2208 (C≡C).

¹⁹F NMR (300 MHz, Gel Phase, CDCl₃) δ −123.7.

¹³C NMR (75 MHz, Gel Phase, CDCl) δ 157.1 (J=238 Hz), 153.9, 150.3, 145.6, 119.5 (J=26 Hz), 117-114, 111.4, 95.7, 94.3, 83.2, 70.8, 56.2.

### Example 38

### 4-(4-Fluoro-2-hydroxyphenylethynyl)-2-methoxyphenoxy-resin 29-MR

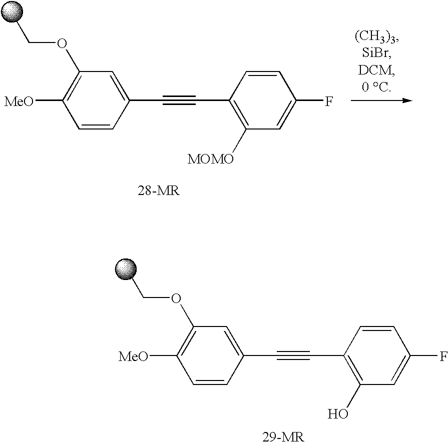

The resin 28-MR (0.11 g, 0.71 mmol/g) was swelled in dry DCM for 30 minutes and (CH₃)₃SiBr (237 mg, 1.55 mmol, 10 equiv.) was added in one portion and was shaken at 0° C. for 8 h. The solid phase was filtered and washed with DCM, DMF, DMF:H₂O (1:1), H₂O and MeOH (5×15 mL, each). The filtered resin was dried under reduce pressure.

### Example 39

### 4-(4-Fluoro-2-iodoacetoxyphenylethynyl)-2-methoxyphenoxy-resin 30-MR

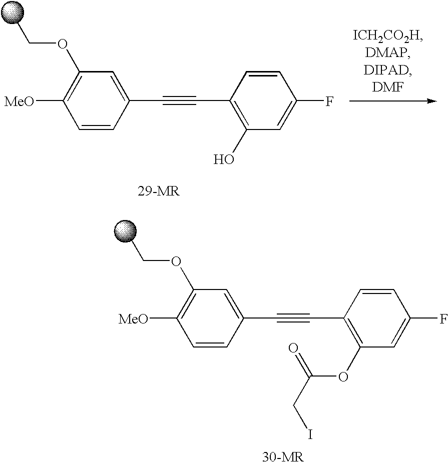

The resin 29-MR (0.18 mg, 0.70 mmol/g) was swelled in 3 mL of dry DMF for 30 minutes. After this time 2-iodoacetic acid (166 mg, 0.89 mmol, 7 equiv.), DMAP (2 mg, 0.02 mmol, 15%) and DIPAD (112 mg, 0.89 mmol, 7 equiv.) were added. The resulting mixture was shaken under Ar for 12 h. The reaction was filtered and the collected solid was washed with DMF, THF, DCM, Et₂O (5×10 mL, each) and finally was dried under reduce pressure. The reaction was repeated under the same conditions to achieve better yields.

### Example 40

### 5-(8,9-Dihydro-11,12-dimethoxy-2-fluoro-6H-[1]benzopyrano[4′,3′:4,5]pyrrolo[2,1-α]isoquinolin-6-one-14-yl)-2-methoxy-phenoxy-resin 31-MR

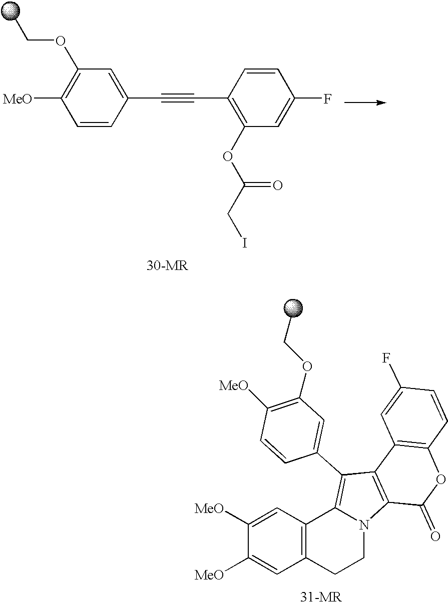

The iodoesther Merrifield resin 30-MR (200 mg, 0.63 mmol/g) was swelled in dry 1,2-dichloroethane (4 mL) and 3,4-dihydro-6,7-dimethoxyisoquinoline 8 (95 mg, 0.5 mmol, 4 equiv.) was added. The reaction mixture was shaken for 24 h at room temperature. After this time DIPEA (65 mg, 4 equiv.) was added and the mixture was heated at 83° C. for 48 h. The reaction mixture was filtered and washed with DCM, DMF, THF, Et₂O (5×5 mL, each). The resin was dried under reduce pressure.

### Example 41

### 8,9-Dihydro-11,12-dimetoxy-2-fluoro-14-(3′-hydroxy-4′-methoxyphenyl)-6H-[1]benzopyrano[4′,3′:4,5]pyrrolo[2,1-α]isoquinolin-6-one 32

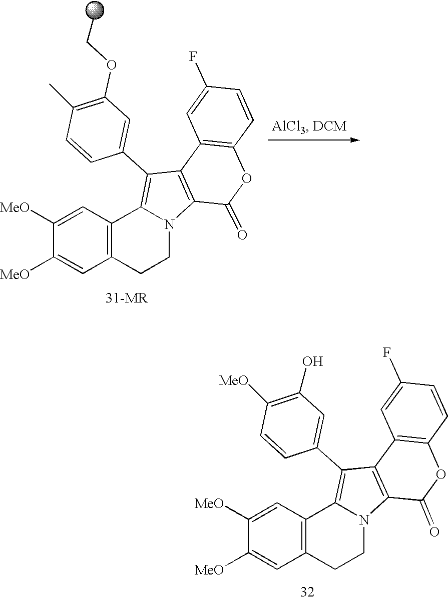

31-MR (185 mg, 0.63 mmol/g loading) was swelled in dry DCM (3 mL) for 30 min and AlCl₃ (67 mg, 0.5 mmol, 4 equiv.) was added. The reaction mixture was stirred in a vibromatic at room temperature for 5 h. After this time was filtered and washed with DCM, EtOAc and MeOH (5×10 mL, each). The combined organic layers were evaporated and re-dissolved in EtOAc. The organic layer was washed with NH₄Cl (sat), (1×20 mL), H₂O (2×20 mL), then the aqueous layer was extracted with EtOAc (2×20 mL). The combined organic layers were dried and evaporated. The HPLC/MS (C18-APCI⁺ using H₂O (5 mM AcNH₄): MeCN gradient 30-100% MeCN in 15 min) shown: lamellarin 32, tr 11.37 min, calcd 487.48, found, 488.2 (M+H)⁺. The crude product was purified by HPLC and lamellarin 32 (3 mg, 6%) was obtained.

¹H NMR (600 MHz, CD₃OD) δ 8.54 (s, 1H), 7.36 (dd, J=9.1 and 4.6 Hz, 1H), 7.19 (d, J=8.6 Hz, 1H), 7.06 (ddd, J=9.1, 7.9 and 3.1 Hz, 1H), 6.93 (dd, J=8.6 Hz, 1H), 6.94 (m, 2 H), 6.70 (s, 1H), 4.75 (m, 2H), 3.97 (s, 3H), 3.84 (s, 3H), 3.33 (s, 3H), 3.13 (t, J=6.2 Hz, 2H)

¹⁹F NMR (400 MHz, CD₃OD) δ −120.3.

(+)-HRMS m/z 488.1506 (calcd for C₂₈H₂₃NO₆F (M+H)⁺ 488.1509, Δ−0.7 ppm).

### Example 42

### 4-(2-Methoxycarbonyl-4-(4isopropoxyphenyl)-1-(triisopropylsilyl)-1H-pyrrol-3-yl)-phenoxy resin 34-MR

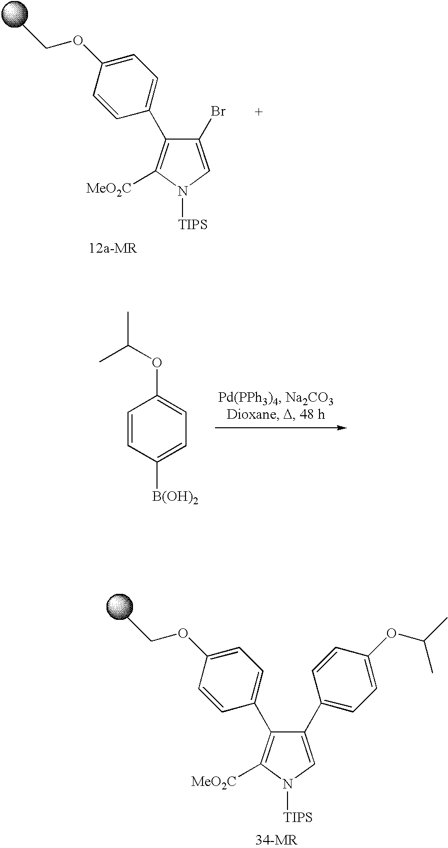

Following the general diaryl synthesis procedure (Example 12), with resin 12a-MR (1 g) and 4-isopropoxyphenylboronic acid gave resin 34-MR after a reaction time of 48 h.

IR (KBr, cm¹) v 1695 (C═O), 1600, 744.

¹³C NMR (CDCl₃, Gel Phase, 75 MHz) δ 13.6 (CH(CH₃)₂), 18.6 (CH(CH₃)₂), 22.1 (OCH(CH₃)₂), 53.4 (CO₂CH₃), 69.6 (OCH(CH₃)₂).

### Example 43

### 4-(2-Methoxycarbonyl-4-(3,4dimethoxyphenyl)-1-(triisopropylsilyl)-1H-pyrrol-3-yl)phenoxy resin 35-MR

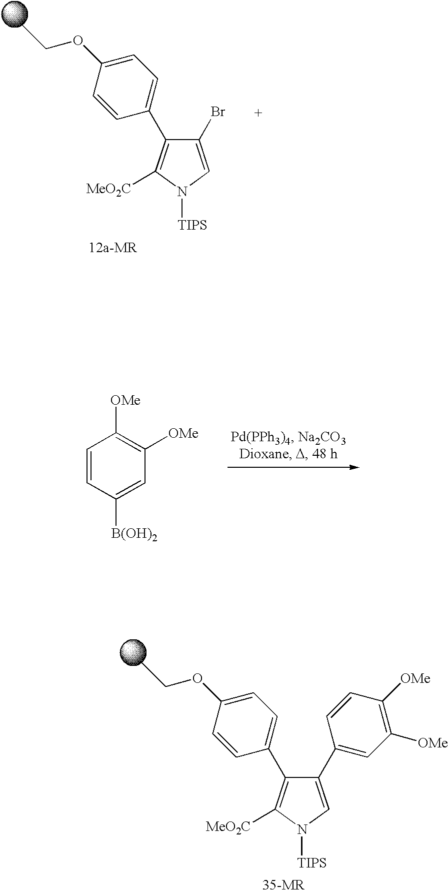

Following the general diaryl synthesis procedure (Example 12), with resin 12a-MR (1 g) and 3,4-dimethoxyphenylboronic acid, gave resin 35-MR after a reaction time of 48 h.

IR (KBr, cm⁻¹) v 1694 (C═O), 1600, 1492, 744.

¹³C NMR (CDCl₃, Gel Phase, 75 MHz) δ 13.6 (CH(CH₃)₂), 18.6 (CH(CH₃)₂), 55.2 (OCH₃), 55.7 (OCH₃).

### Example 44

### 4-(2-Methoxycarbonyl-4-(2-naphthyl)-1-(triisopropylsilyl)-1H-pyrrol-3-yl)-phenoxy resin 36-MR

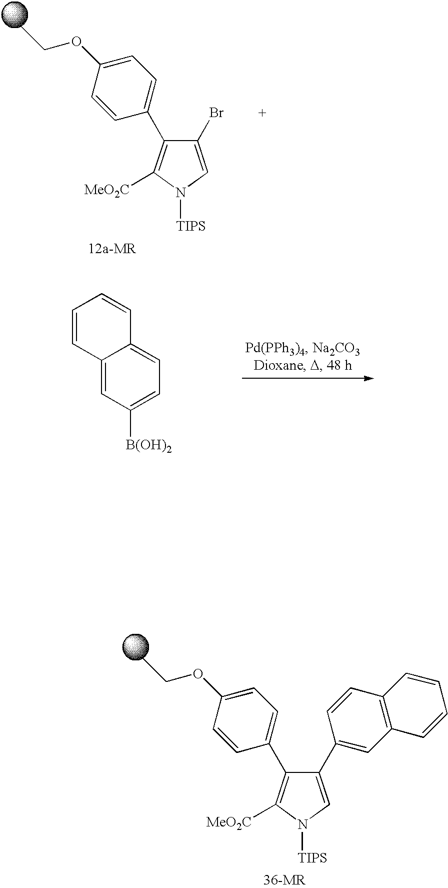

Following the general diaryl synthesis procedure (Example 12), with resin 12a-MR (1 g) and 2-naphthaleneboronic acid, gave resin 36-MR after a reaction time of 48 h.

IR (KBr, cm⁻¹) v 1694 (C═O), 1600.

¹³C NMR (CDCl₃, Gel Phase, 75 MHz) δ 13.6 (CH(CH₃)₂), 18.6 (CH(CH₃)₂).

### Example 45

### Methyl 3,4-bis(4-hydroxyphenyl)-1H-pyrrole-2-carboxylate (Lamellarin Q)

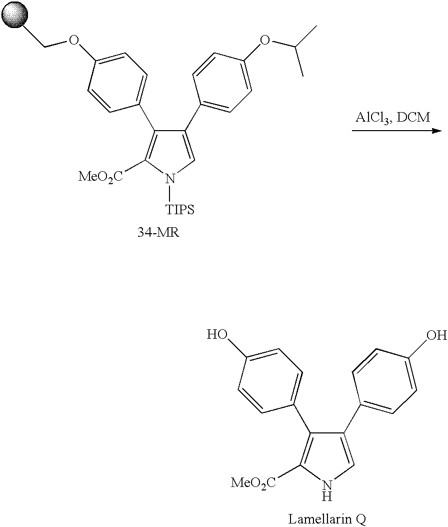

Resin 34-MR (500 mg), gave lamellarin Q by following the general procedure for cleavage with AlCl₃. The crude product was analysed by HPLC-MS (gradient 20-40% MeCN in 15 min) and lamellarin Q (tr 6.92 min, MS 310, M+1) was obtained. Purification by HPLC (gradient 20-40% MeCN in 20 min) gave lamellarin Q (5 mg, 13%) as a pale-yellow gum.

¹H NMR (Acetone-d⁶, 400 MHz) δ 3.64 (s, 3H, CO₂CH₃), 6.66 (d, J=8.4 Hz, 2H, H3′, H5′), 6.76 (d, J=8.4 Hz, 2H, H3″, H5″), 6.95 (d, J=8.4 Hz, 2H, H2″, H6″), 7.05 (d, J=8.4 Hz, 2H, H2″, H6″), 7.13 (bs, 1H, H5), 8.25 (bs, 2H, OH), 10.91 (bs, 1H, NH).

¹³C NMR (Acetone-d⁶, 100 MHz) δ 50.3 (q, CO₂CH₃), 114.4 (d, C3″, C5″), 115.0 (d, C3′, C5′), 120.5 (d, C5), 120.7 (s, C2), 126.1 (s, C4), 126.2 (s, C3), 126.7 (s, C1″), 129.0 (s, C1), 129.5 (d, C2′, C6′), 132.1 (d, C2″, C6″), 155.8 (s, C4′), 156.3 (s, C4″), 161.2 (s, C═O).

MS(EI) m/z 310 (M+1, 25), 309 (M⁺, 22), 278 (100).

HRMS (EI) m/z cacld for C₁₈H₁₅NO₄: 309.1001; found: 309.1012.

### Example 46

### Methyl 3-(4-hydroxyphenyl)-4-(4-isopropoxyphenyl)-1H-pyrrole-2-carboxylate 35

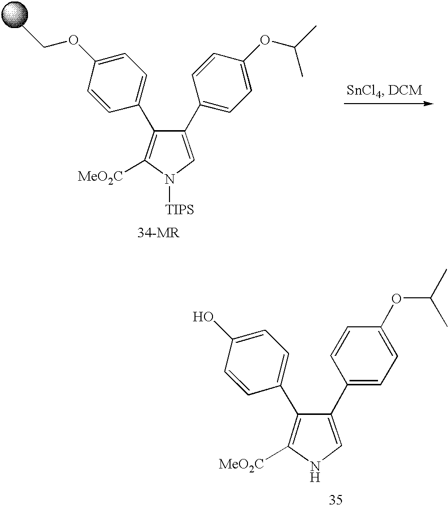

Resin 34-MR (100 mg) was swelled in DCM for 10 min, SnCl₄ (10 equiv.) was added and the mixture was shaken at room temperature for 12 h. The resin was washed with DCM and the organic layer was washed with 10% aq. HCl, dried and evaporated. The crude material was analysed by HPLC-MS (gradient 20-40% MeCN in 15 min) and 35 (tr 10.09 min, MS 352) was obtained. Purification by HPLC (gradient 50-70% MeCN in 20 min) gave 35 (1 mg, 11%) as a white solid.

¹H NMR (Acetone-d⁶, 500 MHz) δ 1.27 (d, J=6.0 Hz, 6H, (CH₃)₂), 3.65 (s, 3H, CO₂CH₃), 4.55 (h, J=6.0 Hz, 1H, CH(CH₃)₂), 6.73 (d, J=8.7 Hz, 2H, H3′, H5′), 6.76 (d, J=8.7 Hz, 2H, H3″, H5″), 7.03 (d, J=8.7 Hz, 2H, H2′, H6′), 7.06 (d, J=8.7 Hz, 2H, H2″, H6″), 7.17 (d, J=3.0 Hz, 1H, H5).

¹³C NMR (Acetone-d⁶, 100 MHz) δ 22.1 (q, (CH₃)₂), 50.3 (q, CO₂CH₃), 69.4 (d, CH(CH₃)₂), 114.5 (d, C3″, C5″), 115.5 (d, C3′, C5′), 120.8 (d, C5), 125.8 (s, C1″), 125.9 (s, C1′),128.8 (s, C3), 129.4 (d, C2′, C6′), 131.8 (s, C4), 132.1 (d, C2″, C6″), 156.1 (s, C4′), 156.3 (s, C4″), 162.6 (s, C═O).

MS (EI) m/z 352 (M+1, 15), 351 (M⁺, 11), 320 (38).

HRMS (EI) m/z cacld for C₂₁H₂₁NO₄: 351.1470; found: 351.1465.

### Example 47

### Methyl 3-(4-hydroxyphenyl)-4-(3,4-dimethoxyphenyl)-1H-pyrrole-2-carboxylate 36

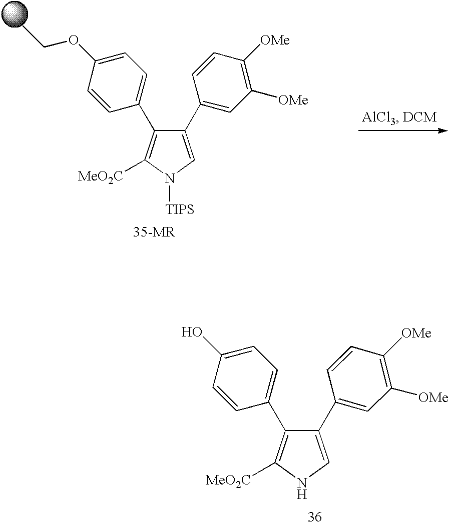

Resin 35-MR (820 mg) gave 36 by following the general procedure for cleavage with AlCl₃ (Example 13). The crude material was analysed by HPLC-MS (gradient 20-40% MeCN in 15 min) and 36 (tr 10.09 min, MS 354, M+1) was obtained. Purification by HPLC (gradient 25-40% MeCN in 30 min) gave 36 (10 mg, 14%) as a white solid.

¹H NMR (Acetone-d⁶, 500 MHz) δ 2.98 (bs, 1H, OH/NH), 3.52 (s, 3H, C4″-OCH₃), 3.65 (s, 3H, CO₂CH₃), 3.75 (s, 3H, C3″-OCH₃), 6.63 (d, J=2.0 Hz, 1H, H2″), 6.77-6.81 (m, 3H, H5′, H3′, H6″), 7.07 (d, J=8.5 Hz, 2H, H6′, H2′), 7.23 (d, J=3.5 Hz, 1H, H5), 10.96 (bs, 1H, NH/OH).

¹³C NMR (Acetone-d⁶, 100 MHz) δ 50.3 (q, CO₂CH₃), 54.9 (q, OCH₃), 55.4 (q, OCH₃), 112.0 (d, C6″), 112.6 (d, C2″), 114.5 (d, C5′, C3′), 119.4 (s, C2), 120.2 (d, C5″), 120.6 (d, C5), 120.8 (s, C4), 126.4 (s, C1″), 128.3 (s, C3), 129.1 (s, C1′), 132.1 (s, C6′, C2′), 147.9 (s, C3′), 149.1 (s, C4″), 156.4 (s, C4′), 161.2 (s, C═O).

MS (EI) m/z 354 (M+1, 39), 353 (M⁺, 31), 322 (100).

HRMS (EI) m/z cacld for C₂₀H₁₉NO₅: 353.1263; found: 353.1261.

### Example 48

### Methyl 3-(4-hydroxyphenyl)-4-(naphthalen-2-yl)-1H-pyrrole-2-carboxylate 37

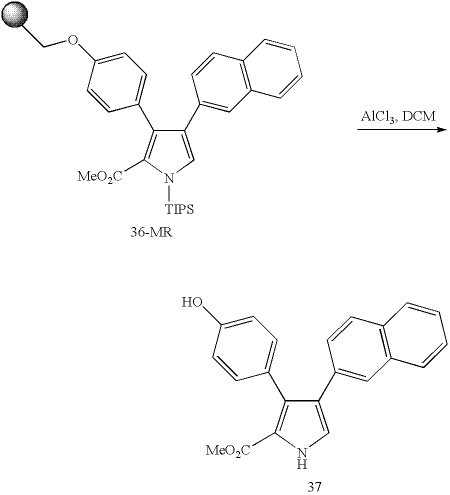

Resin 36-HE (700 mg) gave 37 by following the general procedure for cleavage with AlCl₃ (Example 13). The crude material was analysed by HPLC-MS (gradient 30-70% MeCN in 15 min) and 37 (tr 8.6 min, MS 344, M+1) was obtained. Purification by HPLC (gradient 35-65% MeCN in 30 min) gave 37 (1 mg, 2%) as a white solid.

¹H NMR (Acetone-d⁶, 500 MHz) δ 3.67 (s, 3H, CO₂CH₃), 6.76 (d, J=9.0 Hz, 2H, H3′, H5′), 7.10 (d, J=9.0 Hz, 2H, H2′, H6′), 7.24 (dd, J=8.0 Hz, 1.5, 1H, H3″), 7.40 (m, 3H, H5, H5″, H8″), 7.67 (m, 3H, H1″, H6″, H7″), 7.79 (m, 1H, H4″).

MS (EI) m/z 344 (M+1, 19), 343 (M⁺, 59).

HRMS (EI) m/z cacld for C₂₂H₁₇NO₃: 343.1208; found: 343.1193.

### Example 49

### 4-(2-Methoxycarbonyl-4-(4-isopropoxyphenyl)-1H-pyrrol-3-yl)-phenoxy resin 38-MR

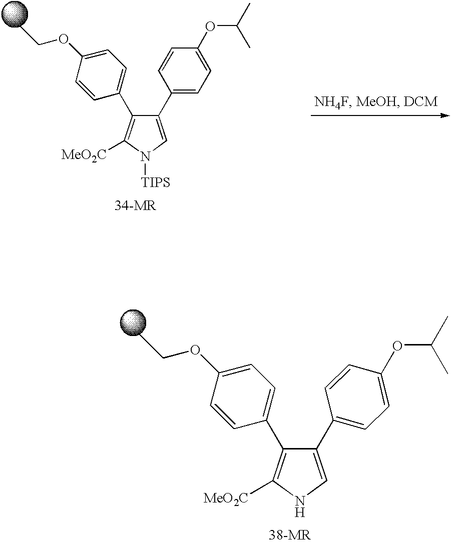

Resin 34-MR (370 mg) gave resin 38-MR by following the procedure for desilylation described in the Example 14.

IR (KBr, cm⁻¹) v 3430, 3287, 1690 (C═O), 1600, 755.

¹³C NMR (CDCl₃, Gel Phase, 75 MHz) δ 22.0 (OCH(CH₃)₂), 53.3 (CO₂CH₃), 69.6 (OCH(CH₃)₂).

### Example 50

### 4-(2-Methoxycarbonyl)-4-(4-methoxyphenyl)-1-(2-(4-methoxyphenyl-2-oxo-ethyl)-1H-pyrrol-3-yl)-phenoxy-resin 17-MR

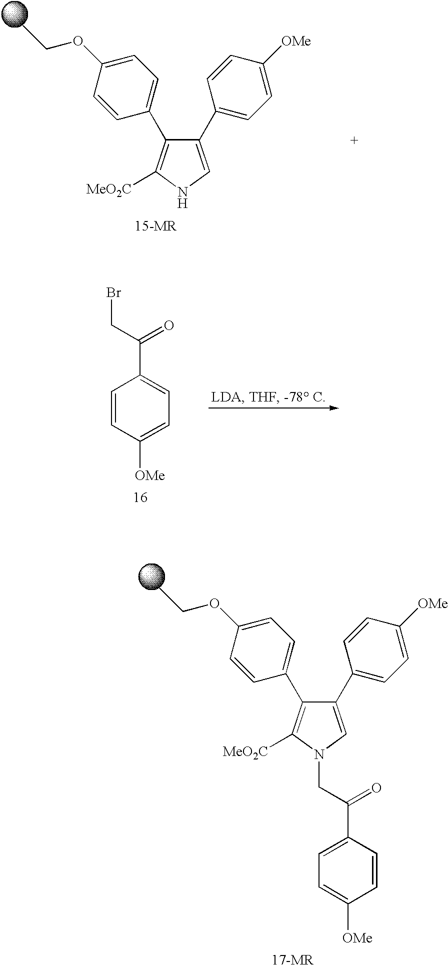

LDA (150 μL, 2 equiv.) was added dropwise to swelled resin 15-MR (370 mg) in dry THF (10 mL) under N₂ at −78° C. The resin was shaken for 1 h at this temperature. 2-Bromo-1-(4-methoxyphenyl)ethanone 16 (172 mg, 5 equiv.) was added. The cooling bath was removed and the crude mixture was shaken in a vibromatic at 86° C. for 24 h. After this time the resin was washed with THF, DCM, MeOH and Et₂O (3×5 mL, each) and dried under vacuum.

IR (KBr, cm⁻¹) v 3400, 2920, 1691, 1600.

¹³C NMR (CDCl₃, 75 MHz) δ 55.2 (CO₂CH₃), 69.9 (OCH₃).

### Example 51

### 4-(2-Methoxycarbonyl-4-(4-isopropoxyphenyl)-1-(4-methoxyphenyl-2-oxo-ethyl)-1H-pyrrol-3-yl)-phenoxy-resin 39-MR

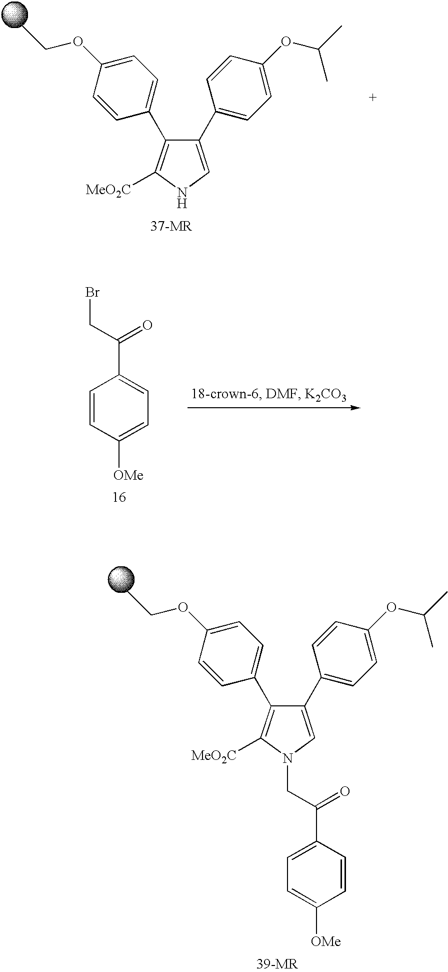

Resin 37-MR (500 mg) was swelled in a solution of 18-crown-6 in DMF (2.5 M, 25 mL) for 10 min. K₂CO₃ (6 equiv.) and 2-bromo-1-(4-methoxyphenyl)ethanone 16 (6 equiv.) were added. The reaction mixture was heated in a microwave oven at 100° C. and 30-40 W during 2 min. The resin was washed with DMF, DMF:H₂O (1:1), DCM, MeOH and Et₂O (3×5 mL, each) and dried under vacuum to afford 39-MR.

IR (KBr, cm⁻¹) v 3417, 1724(C═O), 1690 (C═O), 1600.

¹³C NMR (CDCl₃, 75 MHz) δ 22.1 (OCH(CH₃)₂), 59.8 (CO₂CH₃), 69.8 (OCH(CH₃)₂).

### Example 52

### 4-(2-Methoxycarbonyl-4-(4-isopropoxyphenyl)-1-methyl-1H-pyrrol-3-yl)-phenoxy resin 40-MR

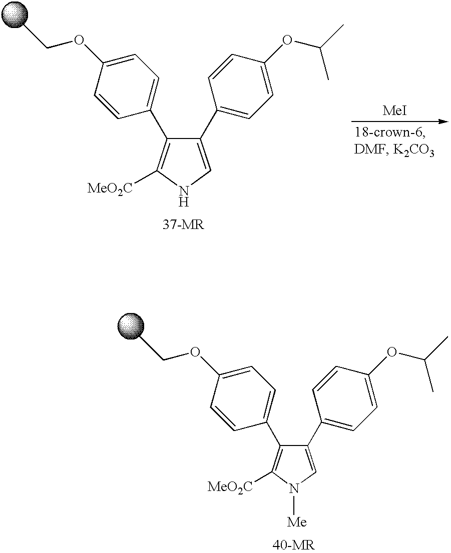

Resin 37-MR (300 mg) was swelled in a solution of 18-crown-6 in DMF (2.5 M) for 10 min and K₂CO₃ (15 equiv.) and MeI (6 equiv.) were added. The reaction mixture was shaken at room temperature for 24 h. The resin was washed with DMF, DMF/H₂O (1:1), DCM, MeOH and Et₂O (3×5 mL, each) and dried under vacuum to afford 40-MR.

IR (KBr, cm⁻¹) v 1693, 1600, 1492, 743.

¹³C NMR (CDCl₃, Gel Phase, 75 MHz) δ 22.1 (OCH(CH₃)₂), 69.6 (OCH(CH₃)₂).

### Example 53

### 4-(2-Methoxycarbonyl-4-(4-isopropoxyphenyl)-1-(2-bromophenethyl)-1H-pyrrol-3-yl)-phenoxy-resin 42-MR

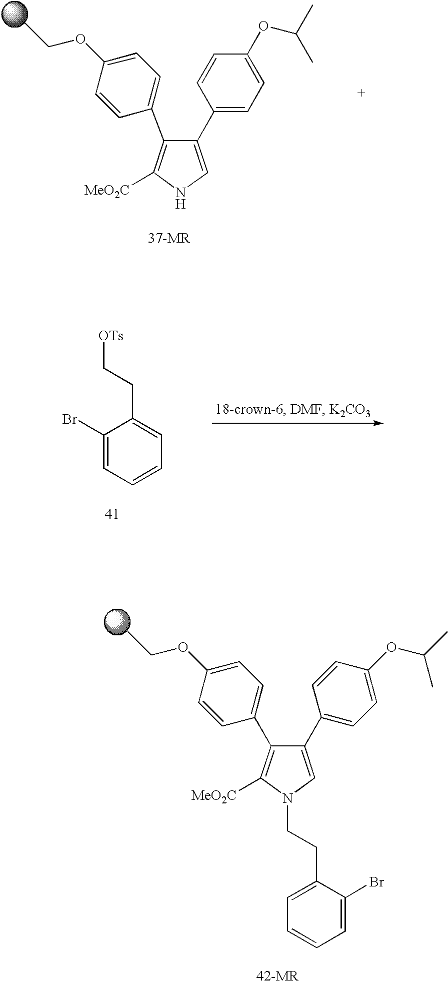

Resin 37-MR (500 mg) was swelled in a solution of 18-crown-6 in DMF (2.5 M, 25 mL) for 10 min and K₂CO₃ (4 equiv.) and 2-bromophenethyl 4-methylbenzenesulfonate 41 (4 equiv.) were added. The reaction mixture was shaken at 80° C. for 24 h. After this time, the resin was washed with DMF, DMF/H₂O (1:1), DCM, MeOH and Et₂O (3×5 mL, each) and dried under vacuum to afford 42-MR.

IR (KBr, cm^{31 1}) v 1642, 610.

¹³C NMR (CDCl₃, 75 MHz) δ 22.1 (OCH(CH₃)₂), 69.6 (OCH(CH₃)₂).

### Example 54

### Methyl 3,4-bis(4-hydroxyphenyl)-1-methyl-1H-pyrrole-2-carboxylate 43

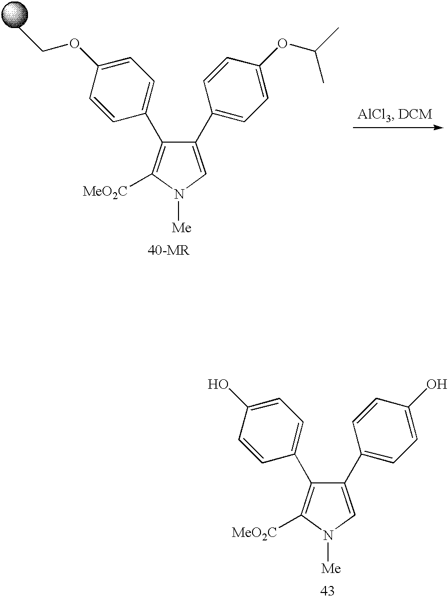

Resin 40-MR (500 mg) gave 43 by following the general procedure for cleavage with AlCl₃ (Example 13). The crude product was analysed by HPLC-MS (gradient 30-70% MeCN in 15 min): 43 (tr 5.67 min, MS=324, M+1). Purification by HPLC (gradient 20-70% MeCN in 50 min) gave 43 (1.7 mg, 6%) as a brown solid.

¹H NMR (Acetone-d⁶, 500 MHz) δ 2.92 (bs, 1H, OH), 3.53 (s, 3H, CO₂CH₃), 3.95 (s, 3H, N—CH₃), 6.64 (d, J=8.5 Hz, 2H, H3′, H5′), 6.75 (d, J=8.5 Hz, 2H, H3″, H5″), 6.91 (d, J=8.5 Hz, 2H, H2′, H6′), 6.97 (d, J=8.5 Hz, 2H, H2″, H6″), 7.10 (s, 1H, H5).

¹³C NMR (Acetone-d⁶, 100 MHz) δ 36.9 (q, CH₃), 50.0 (q, CO₂CH₃), 112.4 (d, C3″, C5″), 115.0 (d, C3′, C5′), 120.6 (s, C2), 124.1 (s, C4), 126.6 (s, C1′), 127.0 (d, C5), 127.4 (s, C1″), 129.4 (d, C2′, C5′), 130.6 (s, C3), 131.9 (d, C2″, C5″), 155.8 (s, C4′), 156.3 (s, C4″), 162.64 (s, C═O).

MS(EI) m/z 324 (M+1, 14), 323 (M⁺, 23), 292 (58).

HRMS (EI) m/z cacld for C₁₉H₁₇NO₄: 323.1158; found 323.1150.

### Example 55

### Methyl 1-(2-bromophenethyl)-3,4-bis(4-hydroxyphenyl)-1H-pyrrole-2-carboxylate 44

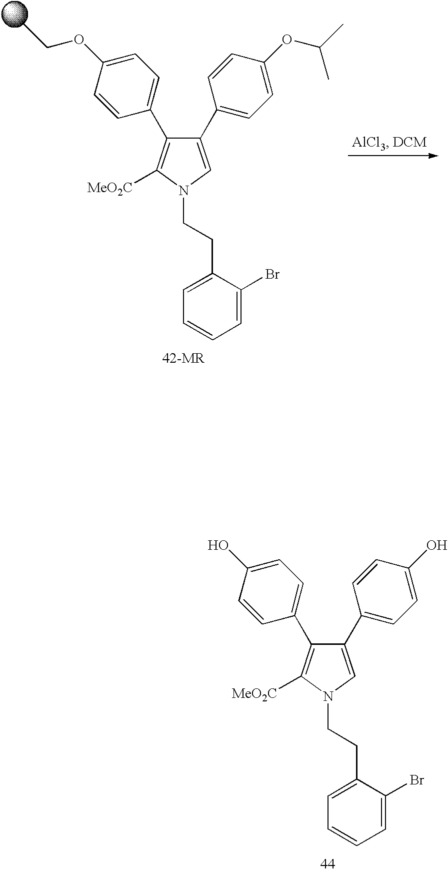

Resin 42-MR (500 mg) gave 44 by following the general procedure for cleavage with AlCl₃ (Example 13). The crude product was analysed by HPLC-MS (gradient 30-70% MeCN in 15 min): 44 (tr 12.64 min, MS=493, M+1).

### Example 56

### Methyl 3,4-bis-(4-hydroxyphenyl)-1-(2-(4-methoxyphenyl)-2-ethanone-yl)-1H-pyrrole-2-carboxylate: lamellarin O

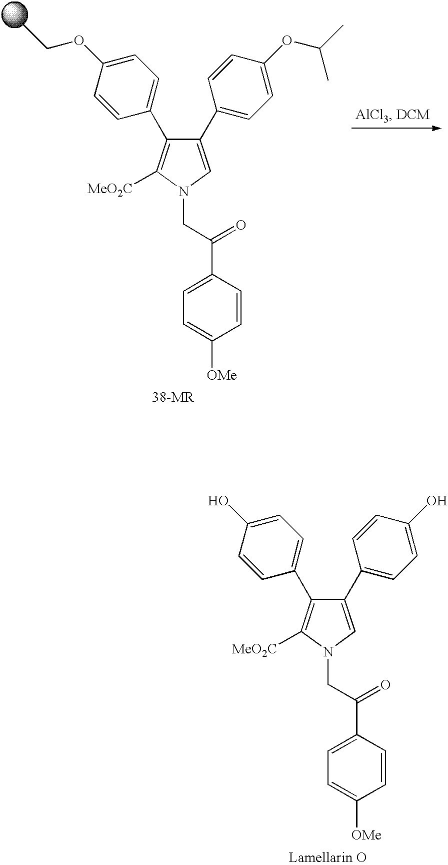

Resin 38-MR (500 mg) gave lamellarin O by following the general procedure for cleavage with AlCl₃ (Example 13). The crude product was analysed by HPLC-MS (gradient 40-80% MeCN in 15 min): lameflarin O (tr 11.94 min, MS=458, M+1).

### Example 57

### 5-(3-Isopropoxy-2,11,12-trimethoxy-6H-[1]benzopyrano[4′,3′:4,5]pyrrolo[2,1-α]isoquinolin-6-one-14-yl)-2-methoxy-phenoxy-resin 45-MR

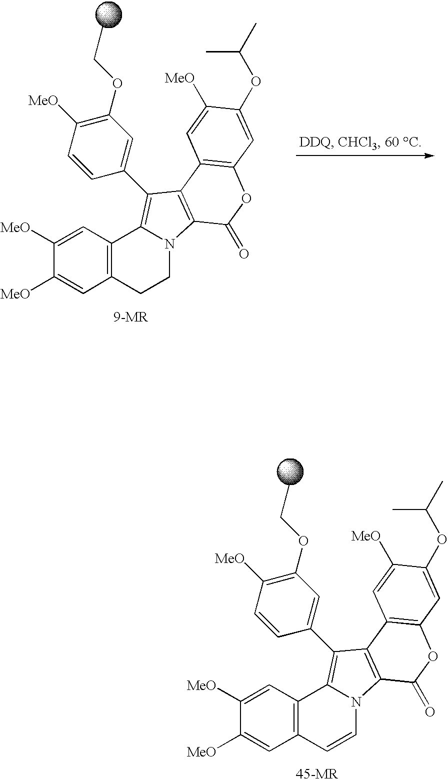

The resin 9-MR (0.4 g, 0.66 mmol/g loading) was swelled in dry CHCl₃ for 30 min. then filtered and swelled again. To the swelled resin with CHCl₃ (5 mL), DDQ (177 mg, 0.78 mmol, 3 equiv.) were added and the resulting mixture was shaken at 55° C. for 5 h. The solvent was then removed. The resin was washed with DMF, DCM, MeOH, and Et₂O (7×5 mL, each). The resin was dried under reduced pressure.

### Example 58

### 3-Acetoxy-2,11,12-trimethoxy-13-(3′-acetoxy-4′-methoxy-phenyl)-6H-[1]benzopyrano[4′,3′:4,5]pyrrolo[2,1-α]isoquinolin-6-one: 3,3′-Di-O-acetyl-12-methoxylamellarin N

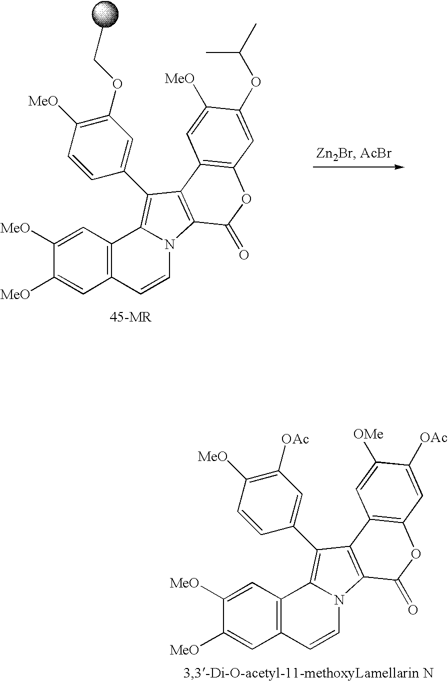

The general procedure described in Example 5 from 45-MR (110 mg, 0.63 mmol/g theoretical loading), dry ZnBr₂ (63 mg, 4 equiv.) and acetylbromide (86 mg, 10 equiv.) was followed. The crude was analysed by HPLC-MS (C18-APCI⁺ using H₂O (5 mM AcNH₄): MeCN gradient 50-100% MeCN in 15 min): tr 10.52 min, calcd for 597.56; found 598.8 (M+H)⁺.

MSMS (Q-TOF) calcd for 597.16; found 597.87 (M+H)⁺, 555.98 ((M+H)⁺—C₂H₂O), 514.09 ((M+H)⁺-2×C₂H₂O).

The crude product was purified by HPLC and 3,3′-Di-O-acetyl-11-methoxylamellarin N (4 mg, 10%) was obtained.

(+)-HRMS m/z 598.1704 (calcd for C₂₉H₂₆NO₈ (M+H)⁺, found 598.1713, Δ−0.9 ppm).

### Example 59

### 3-Hydroxy-2,10,11-trimethoxy-13-(3′-hydroxy-4′-methoxy-phenyl)-6H-[1]benzopyrano[4′,3′:4,5]pyrrolo[2,1-α]isoquinolin-6-one: 11-Methoxylamellarin N, 3,10-dihydroxy-2,12-dimethoxy-14-(3′-hydroxy-4′-methoxy-phenyl)-6H-[1]benzopyrano[4′,3′:4,5]pyrrolo[2,1-α]isoquinolin-6-one: Lamellarin N, and 3,12-dihydroxy-2,11-dimethoxy-13-(3′-hydroxy-4′-methoxy-phenyl)-6H-[1]benzopyrano[4′,3′:4,5]pyrrolo[2,1-α]isoquinolin-6-one: 11-Methoxy-12-demethyllamellarin N

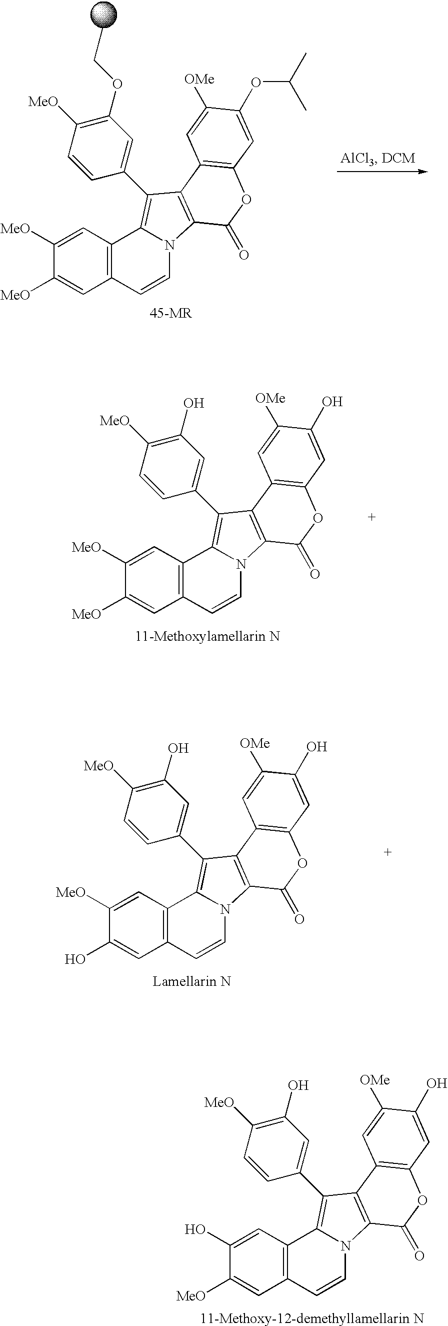

The resin 45-MR (185 mg, 0.63 mmol/g loading) was swelled in dry DCM (3 mL) for 30 min under Ar and AlCl₃ (155 mg, 1.16 mmol, 10 equiv.) was added. The reaction mixture was stirred in a vibromatic shaker at 25° C. for 6 hs. It was then filtered and washed with DCM, EtOAc and MeOH (5×10 mL, each), the organic solvent was evaporated. The residue was taken with a sat. aq. solution of NH₄Cl and extracted with EtOAc (5×20 mL), then washed with brine (1×30 mL). The organic fraction was dried and evaporated.

The HPLC/MS (C18-APCI⁺ using H₂O (5 mM AcNH₄): MeCN gradient 30-100% MeCN in 15 min) shown three Lamellarin derivatives: 11-Methoxylamellarin N: tr 10.75 min, calcd 513.49; found 514.51 (M+H)⁺; 11-Methoxy-12-demethyllamellarin N: tr 9.19 min, calcd 499.47, found 500.42 (M+H)⁺ and Lamellarin N: tr 8.90 min, calcd. 499.47, found 500.42 (M+H)⁺. The crude product was purified by HPLC: 11-Methoxylamellarin N (5 mg, 8%), 11-Methoxy-12-demethyllameliarin N (1 mg, 2%), and Lamellarin N (3 mg, 5%) were obtained.

### 11-Methoxylamellarin N

¹H NMR (600 MHz, CD₃OD) δ 9.12 (d, J=7.2 Hz, 1H), 8.54 (bs, 2H), 7.30 (s, 1H), 7.21 (d, J=7.2 Hz, 1H), 6.85 (s, 1H), 6.84 (s, 1H), 3.97 (s, 3H), 3.94 (s, 3H), 3.49 (s, 3H), 3.46 (s, 3H).

(+)-HRMS m/z 514.1494 (calcd for C₂₉H₂₄NO₈ (M+H)⁺ 514.1502, Δ−1.6 ppm).

### Lamellarin N

¹H NMR (600 MHz, CD₃OD) δ 9.06 (d, J=7.3 Hz, 1H), 7.12 (s, 1H), 7.07 (d, J=1.9 Hz, 1H), 7.05 (s, 1H), 7.05 (d, J=8.2 Hz, 1H), 7.00 (dd, J=8.2 and 1.9 Hz, 1H), 6.91 (s, 1H), 6.86 (s, 1H), 6.70 (s, 1H), 3.91 (s, 3H), 3.43 (s, 6H).

¹³C NMR (150 MHz, CDCl₃)²³ δ 154.1, 147.6, 147.4, 147.2, 147.1, 147.0, 146.5, 143.9, 134.6, 129.48; 128.6, 125.1, 122.9, 122.8, 118.3, 117.9, 112.0, 111.7, 110.7, 109.3, 107.7, 106.5, 105.2, 105.0, 103.3, 55.9, 54.9, 54.9.

(+)-HRMS m/z 500.1348 (calcd for C₂₈H₂₂NO₈ (M+H)⁺ 500.1345, Δ−0.6 ppm).

1. A step for the preparation of a lamellarin, which comprises the reaction: 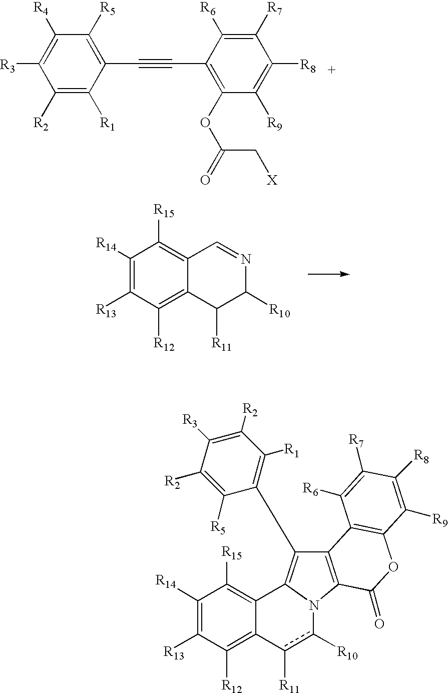
wherein R₁ to R₁₅ are each independently selected from the group consisting of H, halogen, OH, OR′, SH, SR′, SOR′, SO₂R′, NHR′, N(R′)₂, NHCOR′, N(COR′)₂, NHSO₂R′, OC(═O)H, OC(═O)R′, COOH, COOR′, C₁-C₁₂ alkyl, C₁-C₁₂ haloalkyl, C₂-C₁₂ alkenyl, C₂-C₁₂ alkynyl, substituted or unsubstituted aryl, substituted or unsubstituted aralkyl and substituted or unsubstituted heteroaromatic;
wherein each of the R′ groups is independently selected from the group consisting of H, OH, NO₂, NH₂, NHalkyl, N(alkyl)₂, SH, Salkyl, CN, halogen, ═O, C(═O)H, C(═O)alkyl, COOH, COOalkyl, substituted or unsubstituted C₁-C₁₈ alkyl, substituted or unsubstituted C₂-C₁₈ alkenyl, substituted or unsubstituted C₂-C₁₈ alkynyl, substituted or unsubstituted aryl;
the substituents R₁—R₉, R₁₂—R]₅, can form together with an adjacent substituent an aryl, a cycloalkyl or an heterocyclic group;
the dotted line indicates an optional double bond;
with the possible substitution of one or more of the three phenyl rings by a non aromatic heterocyclic ring or by a substituted carbocyclic structure; and
X is halogen;
provided that one of R₂, R₃ or R₄ is immobilised to a resin.

2. A step according to claim 1, which comprises alkylation of a resin compound IV with a 3,4-dihydroisoquinoline V followed by a dipolar (2+3) cycloaddition to afford a resin pentacyclic system: 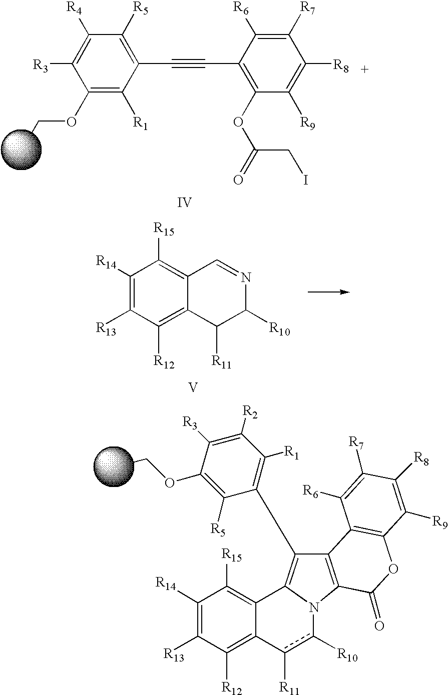
wherein R₁ to R₁₅ and the dotted line are as defined.

3. A step according to claim 1, wherein each of R₁ to R₁₅ is chosen from —H, —OH, -halo, —Oalkyl or —OCOalkyl.

4. A step according to claim 3, where halo is chloro or bromo.

5. A step according to claim 3, wherein alkyl is methyl.

6. A step according to claim 3, wherein at least one of the following is true:
R₁ is H;
R₂ is OH, OMe;
R₃ is OH, OMe;
R₄ is H;
R₅ is H;
R₆ is H;
R₇ is OH, OMe;
R₈ is OH, OMe;
R₉ is H;
R₁₀ is H, OH;
R₁₁ is H;
R₁₂ is H, OH, OMe;
R₁₃ is OH, OMe;
R₁₄ is OH, OMe;
R₁₅ is H.

7. A step according to claim 1, when used as part of a reaction scheme for the preparation of a lamellarin compound (1) of the formula: 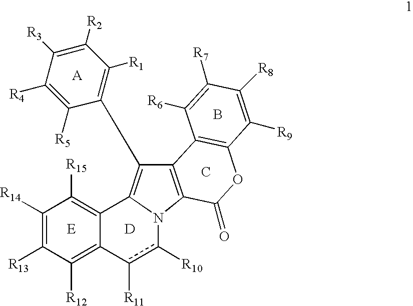
wherein rings A, B and E are aromatic carbocyclic or heterocyclic rings, or with the possible substitution of one or more of these rings by non aromatic heterocyclic rings or substituted carbocyclic structures;
R₁ to R₁₅ and the dotted line are as defined in claim 1.

8. A step according to claim 7, wherein the reaction scheme is as follows: 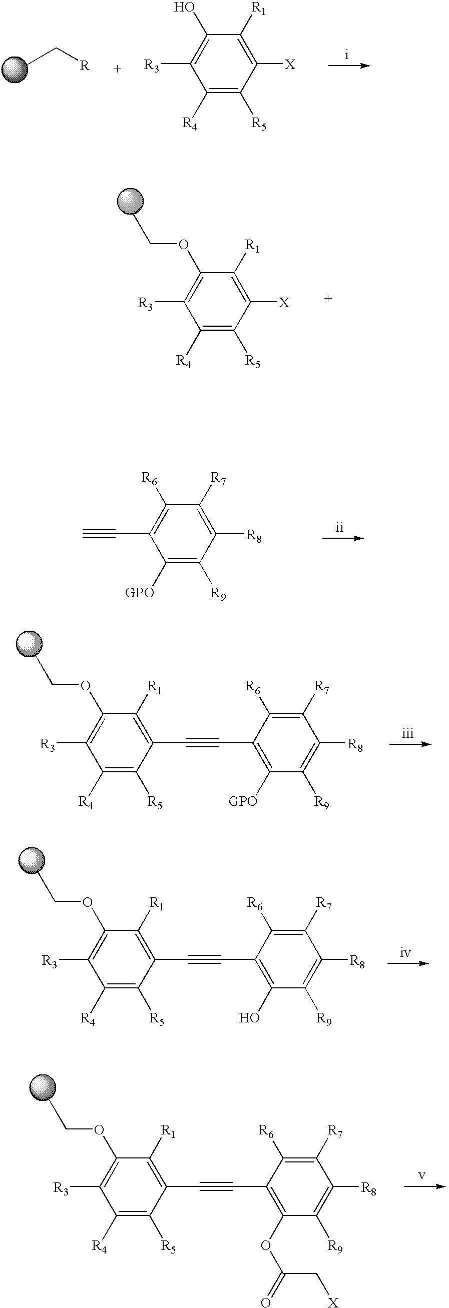 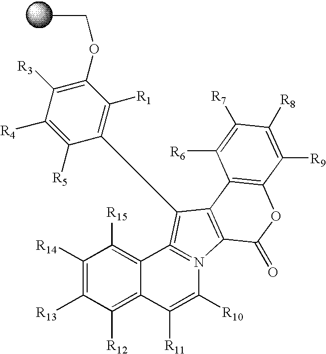
where the solid phase substituent can be replaced by a substituent R₂ and be at the position of R₃ or R₄.

9. A step for the preparation of a lamellarin, which comprises the reaction: 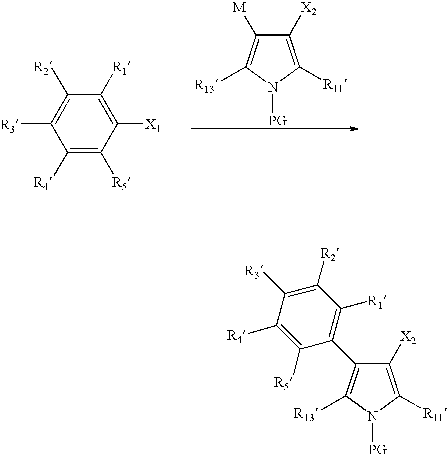
where R₁′ to R₅′, R₁₁′ and R₁₃′are each independently selected from the group consisting of H, halogen, OH, OR′, SH, SR′, SOR′, SO₂R′, NHR′, N(R′)₂, NHCOR′, N(COR′)₂, NHSO₂R′, OC(═O)H, OC(═O)R′, COOH, COOR′, C₁-C₁₂ alkyl, C₁-C₁₂ haloalkyl, C₂-C₁₂ alkenyl, C₂-C₁₂ alkynyl, substituted or unsubstituted aryl, substituted or unsubstituted aralkyl and substituted or unsubstituted heteroaromatic;
wherein each of the R′ groups is independently selected from the group consisting of H, OH, NO₂, NH₂, NHalkyl, N(alkyl)₂, SH, Salkyl, CN, halogen, ═O, C(═O)H, C(═O)alkyl, COOH, COOalkyl, substituted or unsubstituted C₁-C₁₈ alkyl, substituted or unsubstituted C₂-C₁₈ alkenyl, substituted or unsubstituted C₂-C₁₈ alkynyl, substituted or unsubstituted aryl;
with the possible substitution of the phenyl ring by a non aromatic heterocyclic ring or by a substituted carbocyclic structure;
M is a metal function;
X₁ and X₂ are halogen, and
PG is an amino protecting group;
provided that one of that R₂, R₃ or R₄ is immobilised to a resin.

10. A step according to claim 9, which comprises coupling a resin-attached phenol I with 11 to afford the resin VII: 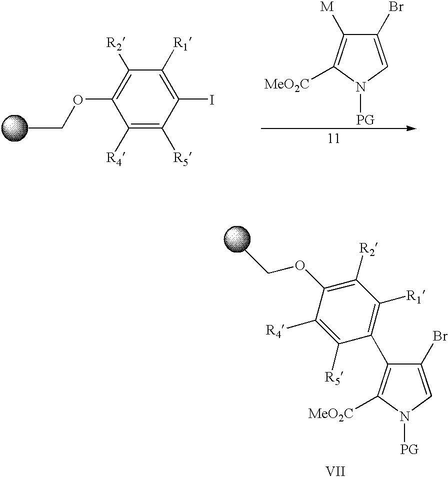
wherein PG, M and R₁ to R₅ are as defined.

11. A step according to claim 9, wherein each of R₁′, R₂′, R₄′, R₅′, R₁₁′, R₁₃′ is chosen from —H, —OH, -halo, —Oalkyl or —COOR′.

12. A step according to claim 11, where halo is chloro or bromo.

13. A step according to claim 11, wherein alkyl is methyl.

14. A step according to claim 11, wherein at least one of the following is true:
R₁′ is H;
R₂′ is H;
R₃′ is OH, OMe;
R₄′ is H;
R₅′ is H;
R₁₁′ is H;
R₁₃′ is H, COOalkyl;

15. A step according to any of claims 9, when used as part of a reaction scheme for the preparation of a lamellarin compound (2) of the formula: 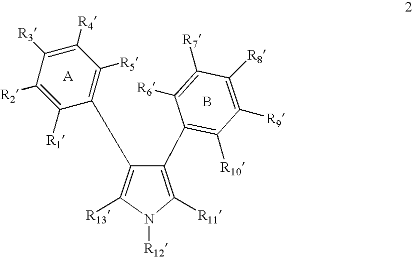
wherein rings A and B are aromatic carbocyclic or heterocyclic rings, or with the possible substitution of one or more of these rings by a non aromatic heterocyclic ring or by a substituted carbocyclic structure;
R₁40 —R₁₁′ and R₁₃′ groups are each independently selected from the group consisting of H, halogen, OH, OR′, SH, SR′, SOR′, SO₂R′, NHR′, N(R′)₂, NHCOR′, N(COR′)₂, NHSO₂R′, OC(═O)H, OC(═O)R′, COOH, COOR′, C₁-C₁₂ alkyl, C₁-C₁₂ haloalkyl, C₂-C₁₂ alkenyl, C₂-C₁₂ alkynyl, substituted or unsubstituted aryl, substituted or unsubstituted aralkyl and substituted or unsubstituted heteroaromatic;
the substituents R₁′ to R₁₀′ can form together with an adjacent substituent an aryl, a cycloalkyl or an heterocyclic group;
R₁₂′ is selected from the group consisting of H, C₁-C₁₂ alkyl, C₁-C₁₂ haloalkyl, C₂-C₁₂ alkenyl,
C₂-C₁₂ alkynyl, substituted or unsubstituted aryl, substituted or unsubstituted arylalkyl and substituted or unsubstituted heteroaromatic;
each of the R′ groups is independently selected from the group consisting of H, OH, NO₂, NH₂, NHalkyl, N(alkyl)₂, SH, Salkyl, CN, halogen, ═O, C(═O)H, C(═O)alkyl, COOH, COOalkyl, substituted or unsubstituted C₁-C₁₈ alkyl, substituted or unsubstituted C₂-C₁₈ alkenyl, substituted or unsubstituted C₂-C₁₈ alkynyl, substituted or unsubstituted aryl; and
wherein the dotted line means an optional double bond.

16. A step according to claim 15, where the reaction scheme is as follows: 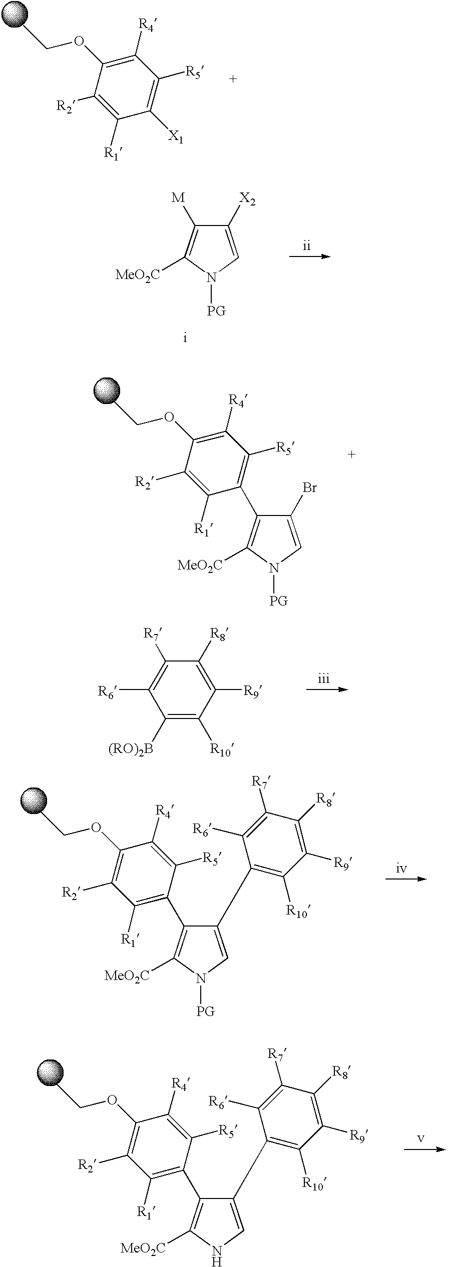 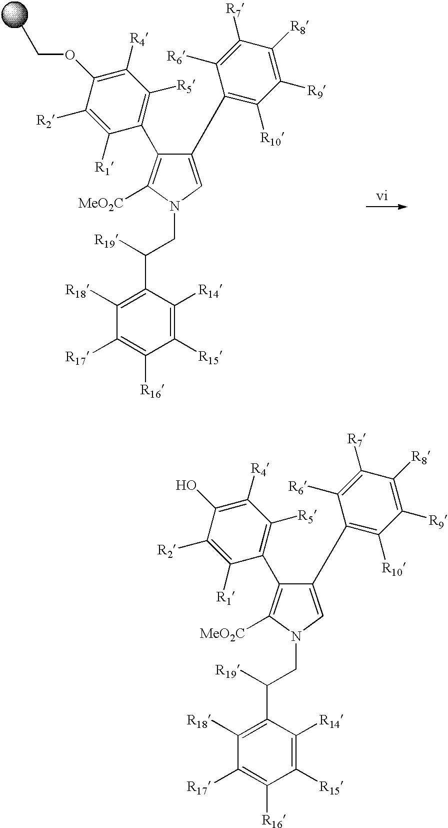
where the solid phase substituent can be replaced by a substituent R₃′ and be at the position of R₂′ or R₄′; R₁′ to R₁₀′, R₁₄′ to R₁₉′, X₁, X₂, M and PG are as defined.
